(19) United States
(12) Patent Application Publication
(10) Pub. No.: US 2025/0312149 A1
(43) Pub. Date: Oct. 9, 2025

(54) IMPLANT WITH SHAPE-CONFORMING ELEMENT

(71) Applicant: CARDIOVALVE LTD., Or Yehuda (IL)

(72) Inventors: Ilia HARITON, Zichron Yaakov (IL); Meni IAMBERGER, Kfar Saba (IL); Hila YARON BRAUMAN, Herzliya (IL); Aviram BAUM, Tel Aviv (IL); Yelena KASIMOV, Petach Tikva (IL)

(73) Assignee: CARDIOVALVE LTD., Or Yehuda (IL)

(21) Appl. No.: 18/872,367

(22) PCT Filed: Jun. 7, 2023
(86) PCT No.: PCT/IL2023/050587

§ 371 Date: Dec. 6, 2024

Related U.S. Application Data
(60) Provisional application No. 63/350,193, filed on Jun. 8, 2022.

(51) Int. Cl. A61F 2/24

(52) U.S. Cl. CPC A61F 2/2418; A61F 2210/0014

(57) ABSTRACT

Apparatus is provided for use at a calcified native valve of a patient's heart, the apparatus including a prosthetic valve (22, 62, or 66) deliverable to the heart through a catheter. The prosthetic valve includes a frame (24) that includes: (i) a valve body (26) that circumscribes a central longitudinal axis of the prosthetic valve and defines a lumen along the axis; and (ii) arms (28) that are attached to and extend from respective circumferential sites of the valve body in an upstream direction. Each of the arms extends radially outward to a respective arm-tip (29) in an expanded state of the frame. Prosthetic leaflets (55) are disposed within the lumen and facilitate one-way fluid flow through the lumen following implantation of the prosthetic valve in the heart. A shape-conforming element (40) surrounds the valve body and conforms to calcified areas of cardiac tissue. Other embodiments are also described.

### CROSS-REFERENCE TO RELATED APPLICATIONS

The present application claims the benefit of U.S. Provisional Patent Application 63/350,193 to Hariton et al., entitled, “Implant with shape-conforming element,” filed Jun. 8, 2022, which is assigned to the assignee of the present patent application and is incorporated herein by reference.

### FIELD OF THE INVENTION

Some applications of the present invention relate in general to valve replacement. More specifically, some applications of the present invention relate to prosthetic valves for replacement of a cardiac valve.

### BACKGROUND

Mitral annular calcification is a chronic process in which there is deposition of calcium in the mitral valve annulus. In mitral annular calcification, the mitral valve annulus becomes less flexible and thicker. Ischemic heart disease causes regurgitation of a heart valve by the combination of ischemic dysfunction of the papillary muscles, and the dilatation of the ventricle that is present in ischemic heart disease, with the subsequent displacement of the papillary muscles and the dilatation of the valve annulus.

Dilation of the annulus of the valve prevents the valve leaflets from fully coapting when the valve is closed. Regurgitation of blood from the ventricle into the atrium results in increased total stroke volume and decreased cardiac output, and ultimate weakening of the ventricle secondary to a volume overload and a pressure overload of the atrium.

### SUMMARY OF THE INVENTION

For some applications of the invention, a prosthetic valve is provided for transluminal implantation of the prosthetic valve at a native valve (e.g., a native heart valve) of a patient, e.g., typically via a catheter. Typically, the prosthetic valve is configured for implantation at a native valve affected by calcification. The prosthetic valve comprises a frame that comprises a valve body that circumscribes a central longitudinal axis of the prosthetic valve and defines a lumen along the axis. The prosthetic valve comprises a plurality of prosthetic leaflets, disposed within the lumen, and arranged to facilitate one-way upstream-to-downstream fluid flow through the lumen following implantation of the prosthetic valve in the heart. A plurality of arms are attached to and extend from respective circumferential sites of the valve body in an upstream direction. Each of the arms is configured to extend radially outward to a respective arm-tip in an expanded state of the frame. The prosthetic valve is configured to be placed at the native valve, such as by placing the plurality of arms of the prosthetic valve against an upstream surface of the native valve (e.g., against a native valve annulus). The prosthetic valve is subsequently implanted at the native valve by expanding the prosthetic valve in an opening defined by the native valve.

The prosthetic valve is typically configured to be implanted at a calcified atrioventricular valve, e.g., a mitral valve or a tricuspid valve. The scope of the present invention includes the prosthetic valve being configured to be implanted at the aortic valve or at the pulmonary valve.

The prosthetic valve comprises a shape-conforming element surrounding the valve body. The shape-conforming element is configured to conform to calcified areas of cardiac tissue of the calcified native heart valve or to apparatus (e.g., an annuloplasty ring or a prosthetic valve) already implanted at the native valve and function as a cushion. That is, portions of the shape-conforming element are deformable to accommodate the tissue at the native valve. Typically, the deformation of the shape-conforming element is configured to enhance anchoring of the prosthetic valve to the native valve or to apparatus already implanted at the native valve. Some portions of the shape-conforming element are deformed by being radially compressed toward a central longitudinal axis of the prosthetic valve so as to conform to radially-inwardly protruding calcified tissue. Some portions of the shape-conforming element fill gaps created between the prosthetic valve and calcified tissue or apparatus already implanted at the native valve. For some applications, the shape-conforming element comprises a foam, a sponge, and/or a fabric. For some applications, the shape-conforming element comprises metal. For some applications, metal such as nitinol and/or stainless steel may be incorporated within the shape-conforming element. For some applications, an expandable element, e.g., a stent, surrounds the shape-conforming element.

For applications in which the shape-conforming element comprises a metal structure, the shape-conforming element may comprise a braided or woven mesh of metal or a stent structure which functions as a cushion. The metal shape-conforming element may be defined by a wall that surrounds a hollow space in a manner in which the braided mesh structure is deformable.

For some applications of the present invention, the prosthetic valve is implantable directly within the calcified native valve. For some applications of the present invention, the prosthetic valve is couplable to a ring, e.g., an annuloplasty ring, that is implantable or has already been implanted at the defective or malfunctioning native valve. For some applications of the present invention, the prosthetic valve is couplable to a prosthetic valve that has already been implanted at the defective or malfunctioning native valve. In such cases, the pre-implanted prosthetic valve may comprise a prosthetic valve that is not functioning properly.

In either application, the shape-conforming element is configured to enhance the adaptability and anchoring of the prosthetic valve to the geometry and topography of the calcified valve or to pre-implanted apparatus at the defective or malfunctioning native valve.

There is therefore provided, in accordance with some applications of the invention apparatus for use at a calcified native valve of a heart of a patient, the apparatus including a prosthetic valve deliverable to the heart through a catheter, the prosthetic valve including:
(a) a frame that includes:
   a valve body that circumscribes a central longitudinal axis of the prosthetic valve and defines a lumen along the axis; and
   a plurality of arms that are attached to and extend from respective circumferential sites of the valve body in an upstream direction, each of the arms configured to extend radially outward to a respective arm-tip in an expanded state of the frame;
(b) a plurality of prosthetic leaflets, disposed within the lumen, and arranged to facilitate one-way upstream-to-downstream fluid flow through the lumen following implantation of the prosthetic valve in the heart; and
(c) a shape-conforming element surrounding the valve body, the shape-conforming element configured to conform to calcified areas of cardiac tissue of the calcified native heart valve.

There is additionally provided, in accordance with some applications of the invention, apparatus including a prosthetic valve deliverable through a catheter to a native valve of a heart of a patient, the prosthetic valve including:
(a) a frame that includes:
   a valve body that circumscribes a central longitudinal axis of the prosthetic valve and defines a lumen along the axis; and
   a plurality of arms that are attached to and extend from respective circumferential sites of the valve body in an upstream direction, each of the arms configured to extend radially outward to a respective arm-tip in an expanded state of the frame;
(b) a plurality of prosthetic leaflets, disposed within the lumen, and arranged to facilitate one-way upstream-to-downstream fluid flow through the lumen following implantation of the prosthetic valve in the heart; and
(c) a shape-conforming element surrounding the valve body, the shape-conforming element being configured to conform to apparatus previously implanted at the native heart valve.

In an application, the shape-conforming element includes a wall surrounding a hollow space.

In an application, the shape-conforming element includes a metal wall surrounding a hollow space.

In an application, the shape-conforming element includes a stent.

In an application, the shape-conforming element includes a braided mesh.

In an application, the plurality of arms includes 3-24 arms.

In an application, each arm, when extending radially outward from the valve body, has a radially-extending portion having a downstream surface having a downstream-directed convex portion adjacent the arm-tip.

In an application, in the expanded state of the frame, (1) each arm has a downstream-directed concave portion at a radially-inner portion of the arm, and (2) the downstream-directed convex portion of each arm is between the downstream-directed concave portion and the arm-tip.

In an application, the downstream-directed convex portion is upstream of an upstream end of the shape-conforming element.

In an application, an upstream end of the shape-conforming element is upstream of the circumferential sites.

In an application, the circumferential sites are between an upstream end and a downstream end of the shape-conforming element.

In an application, the shape-conforming element is configured to enhance friction between the prosthetic valve and the calcified native heart valve.

In an application, the shape-conforming element includes a sponge.

In an application, the shape-conforming element includes polyurethane.

In an application, the shape-conforming element includes a foam.

In an application, the shape-conforming element is not inflatable.

In an application, the shape-conforming element has a thickness of 3-7 mm, measured from a radially innermost portion of the shape-conforming element to a radially outermost portion of the shape-conforming element.

In an application, the shape-conforming element has a thickness of 5 mm, measured from a radially innermost portion of the shape-conforming element to a radially outermost portion of the shape-conforming element.

In an application, the shape-conforming element includes a shape memory polymer.

In an application, the shape-conforming element undergoes a radial deformation of between 1 and 9 mm.

In an application, the shape-conforming element undergoes a radial deformation of 5 mm.

In an application, the shape-conforming element has a pore size of 0.2-3.0 mm.

In an application, the shape-conforming element has a density of 0.01-0.06 g/cm\hat{ }3.

In an application, the shape-conforming element has an ultimate tensile strength of 50-250 kPa.

In an application, the shape-conforming element has a strain at break of 20-80%.

In an application, the shape-conforming element has a glass transition temperature range occurring when the shape-conforming element is wet, between 20-40 degrees C.

In an application, the shape-conforming element has a glass transition temperature range occurring when the shape-conforming element is dry, between 50-70 degrees C.

In an application, the shape-conforming element has a height of 5-20 mm measured along the central longitudinal axis of the prosthetic valve.

In an application, the shape-conforming element covers between 15-95% of the valve body.

In an application, the shape-conforming element is shaped so as to define a plurality of windows through a wall of the shape-conforming element.

In an application, each window has a longest dimension of 2-7 mm, in the expanded state of the frame.

In an application, the shape-conforming element is shaped so as to define 3-18 of the windows through the wall of the shape-conforming element.

In an application, the prosthetic valve is configured to be positioned at an aorta.

In an application, the prosthetic valve is configured treat aortic stenosis.

In an application, the prosthetic valve further includes an outer flexible structure surrounding the shape-conforming element and configured to enhance friction between the prosthetic valve and the calcified native heart valve.

In an application, the outer flexible structure is disposed around the shape-conforming element prior to delivery of the prosthetic valve to the heart of the patient.

In an application, the outer flexible structure includes a stent structure.

In an application, the outer flexible structure includes a tubular structure.

In an application, the outer flexible structure includes a mesh.

In an application, the prosthetic valve further includes a fabric covering surrounding the shape-conforming element and configured to prevent migration of pieces of the shape-confirming material away from the valve body.

In an application, at least one surface of the shape-conforming element is arranged in an undulating pattern around an outer surface of the valve body.

In an application, at least one surface of the shape-conforming element is arranged in a zig-zag pattern around an outer surface of the valve body.

In an application, the at least one surface of the shape-conforming element includes:
an upstream surface of the shape-conforming element that is arranged in a zig-zag pattern; and
a downstream surface of the shape-conforming element that is arranged in a zig-zag pattern.

In an application, the zig-zag pattern of the upstream surface is rotationally offset with respect to the zig-zag pattern of the downstream surface.

In an application, the shape-conforming element is shaped so as to define:
an upstream end having a plurality of upstream surfaces alternating with a plurality of downstream-directed cutout portions, each downstream-directed cutout portion having respective descending and ascending edges connected at a downstream-directed vertex, and
a downstream end having a plurality of downstream surfaces alternating with a plurality of upstream-directed cutout portions, each upstream-directed cutout portion having respective ascending and descending edges connected at an upstream-directed vertex.

In an application, the upstream surfaces of the upstream end and the downstream surfaces of the downstream end are straight.

In an application, the downstream surfaces are offset with respect to the upstream surfaces and the downstream-directed vertices are offset with respect to the upstream-directed vertices.

In an application:
each downstream-directed vertex faces a midpoint of an opposite downstream surface of the downstream end of the shape-conforming element, and
each upstream-directed vertex faces a midpoint of an opposite upstream surface of the upstream end of the shape-conforming element.

There is further provided, in accordance with some applications of the invention, apparatus including a prosthetic valve deliverable through a catheter to a native valve of a heart of a patient, the prosthetic valve including:
(a) a frame that includes:
   a valve body that circumscribes a central longitudinal axis of the prosthetic valve and defines a lumen along the axis; and
   a plurality of arms that are attached to and extend from respective circumferential sites of the valve body in an upstream direction, each of the arms configured to extend radially outward to a respective arm-tip in an expanded state of the frame;
(b) a plurality of prosthetic leaflets, disposed within the lumen, and arranged to facilitate one-way upstream-to-downstream fluid flow through the lumen following implantation of the prosthetic valve in the heart; and
(c) a shape-conforming element surrounding the valve body, the shape-conforming element having a first cross-sectional shape in a resting state and being deformable into a second cross-sectional shape to adapt to a shape of tissue at the native heart valve, in a deployed state of the prosthetic valve,
the shape-conforming element is mechanically isolated from the frame such that the frame maintains an undeformed shape when the shape-conforming element has deformed into the second cross-sectional shape.

There is yet further provided, in accordance with some applications of the invention, apparatus including a prosthetic valve deliverable through a catheter to a native valve of a heart of a patient, the prosthetic valve including:
(a) a frame that includes:
   a valve body that circumscribes a central longitudinal axis of the prosthetic valve and defines a lumen along the axis; and
   a plurality of arms that are attached to and extend from respective circumferential sites of the valve body in an upstream direction, each of the arms configured to extend radially outward to a respective arm-tip in an expanded state of the frame;
(b) a plurality of prosthetic leaflets, disposed within the lumen, and arranged to facilitate one-way upstream-to-downstream fluid flow through the lumen following implantation of the prosthetic valve in the heart; and
(c) a shape-conforming element surrounding the valve body, the shape-conforming element having a first cross-sectional shape in a resting state and being deformable into a second cross-sectional shape to adapt to a shape of apparatus implanted at tissue at the native heart valve, in a deployed state of the prosthetic valve,
the shape-conforming element is mechanically isolated from the frame such that the frame maintains an undeformed shape when the shape-conforming element has deformed into the second cross-sectional shape.

In an application, the shape-conforming element includes a wall surrounding a hollow space.

In an application, the shape-conforming element includes a metal wall surrounding a hollow space.

In an application, the shape-conforming element includes a stent.

In an application, the shape-conforming element includes a braided mesh.

In an application, the shape-conforming element includes a sponge.

In an application, the shape-conforming element includes polyurethane.

In an application, the shape-conforming element includes a foam.

In an application, the shape-conforming element is not inflatable.

In an application, the shape-conforming element includes a shape memory polymer.

In an application, the shape-conforming element undergoes a radial deformation of between 1 and 9 mm.

In an application, the shape-conforming element undergoes a radial deformation of 5 mm.

In an application, the shape-conforming element has a pore size of 0.2-3.0 mm.

In an application, the shape-conforming element has a density of 0.01-0.06 g/cm\hat{ }3.

In an application, the shape-conforming element has an ultimate tensile strength of 50-250 kPa.

In an application, the shape-conforming element has a strain at break of 20-80%.

In an application, the shape-conforming element has a glass transition temperature range occurring when the shape-conforming element is wet, between 20-40 degrees C.

In an application, the shape-conforming element has a glass transition temperature range occurring when the shape-conforming element is dry, between 50-70 degrees C.

There is still further provided, in accordance with some applications of the invention, apparatus including a prosthetic valve deliverable through a catheter to a native valve of a heart of a patient, the prosthetic valve including:
(a) a frame that includes:
   a valve body that circumscribes a central longitudinal axis of the prosthetic valve and defines a lumen along the axis; and
   a plurality of arms that are attached to and extend from respective circumferential sites of the valve body in an upstream direction, each of the arms configured to extend radially outward to a respective arm-tip in an expanded state of the frame;
(b) a plurality of prosthetic leaflets, disposed within the lumen, and arranged to facilitate one-way upstream-to-downstream fluid flow through the lumen following implantation of the prosthetic valve in the heart; and
(c) a metal deformation element surrounding the valve body, the metal deformation element having a first cross-sectional shape in a resting state and being deformable into a second cross-sectional shape to adapt to a shape of tissue at the native heart valve, in a deployed state of the prosthetic valve,
the metal deformation element is mechanically isolated from the frame such that the frame maintains an undeformed shape when the metal deformation element has deformed into the second cross-sectional shape.

There is also provided, in accordance with some applications of the invention, apparatus including a prosthetic valve deliverable through a catheter to a native valve of a heart of a patient, the prosthetic valve including:
(a) a frame that includes:
   a valve body that circumscribes a central longitudinal axis of the prosthetic valve and defines a lumen along the axis; and
   a plurality of arms that are attached to and extend from respective circumferential sites of the valve body in an upstream direction, each of the arms configured to extend radially outward to a respective arm-tip in an expanded state of the frame;
(b) a plurality of prosthetic leaflets, disposed within the lumen, and arranged to facilitate one-way upstream-to-downstream fluid flow through the lumen following implantation of the prosthetic valve in the heart; and
(c) a metal deformation element surrounding the valve body, the metal deformation element having a first cross-sectional shape in a resting state and being deformable into a second cross-sectional shape to adapt to a shape of apparatus implanted at tissue at the native heart valve, in a deployed state of the prosthetic valve,
the metal deformation element is mechanically isolated from the frame such that the frame maintains an undeformed shape when the metal deformation element has deformed into the second cross-sectional shape.

The present invention will be more fully understood from the following detailed description of applications thereof, taken together with the drawings, in which:

### BRIEF DESCRIPTION OF THE DRAWINGS

FIG. 1A is a schematic illustration of a shape-conforming prosthetic valve for implantation at a native valve, in accordance with some applications of the invention;

FIG. 1B is a schematic illustration of another shape-conforming prosthetic valve for implantation at a native valve, in accordance with some applications of the invention;

FIG. 1C is a schematic illustration of yet another shape-conforming prosthetic valve for implantation at a native valve, in accordance with some applications of the invention;

FIGS. 2A-C are schematic illustrations of different shape-conforming elements that surround a valve body of the prosthetic valves of FIGS. 1A-B, in accordance with some applications of the invention;

FIGS. 3A-C are schematic illustrations of different prosthetic valves comprising the respective shape-conforming elements of FIGS. 2A-C, in accordance with some applications of the invention;

FIGS. 4A-B are schematic illustrations of a method of implanting the prosthetic valve at a calcified native valve, in accordance with some applications of the invention;

FIGS. 5A-B are schematic illustrations of the prosthetic valve implanted at a calcified native valve, in accordance with some applications of the invention;

FIGS. 6A-B and 7 are schematic illustrations of different prosthetic valves comprising respective shape-conforming elements, in accordance with some applications of the invention;

FIG. 8 is a schematic illustration of a prosthetic valve conforming to a pre-implanted annuloplasty structure, in accordance with some applications of the invention;

FIGS. 9A-B are schematic illustrations of a prosthetic valve conforming to a pre-implanted prosthetic valve, in accordance with some applications of the invention;

FIG. 10 is a schematic illustration of another prosthetic valve for implantation at a calcified native valve, in accordance with some applications of the invention; and

FIG. 11 is a schematic illustration of implanting the prosthetic valve at a calcified native aortic valve, in accordance with some applications of the invention.

### DETAILED DESCRIPTION OF EMBODIMENTS

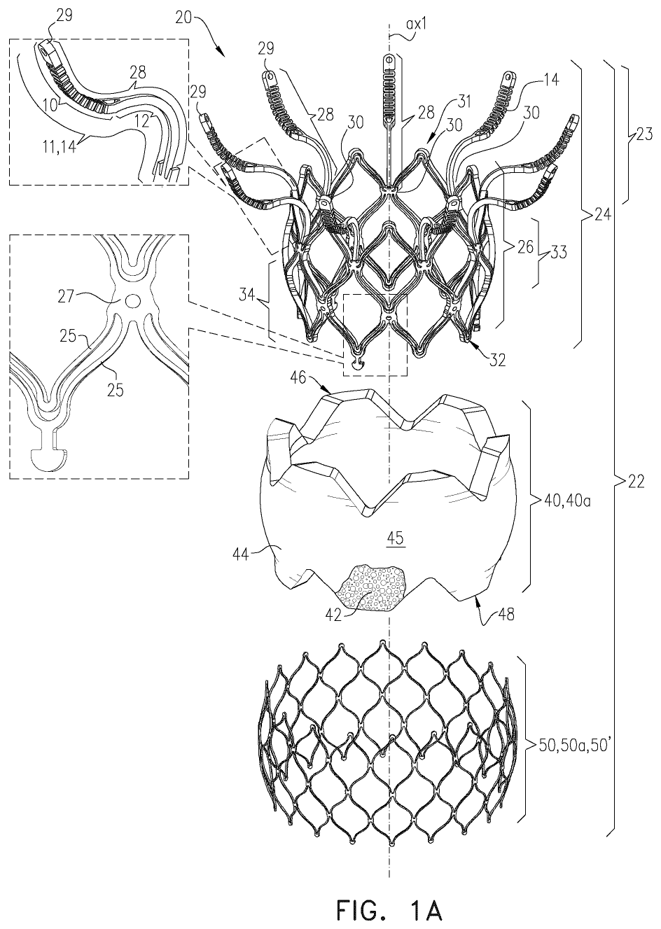

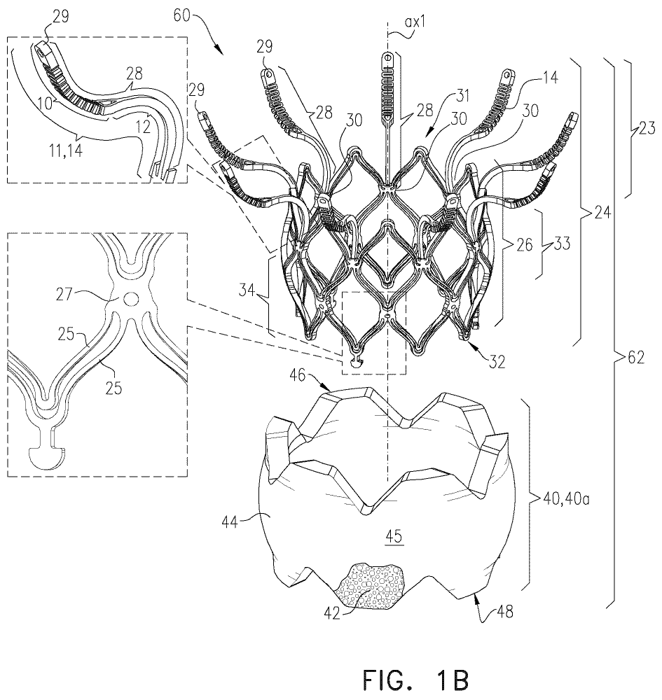

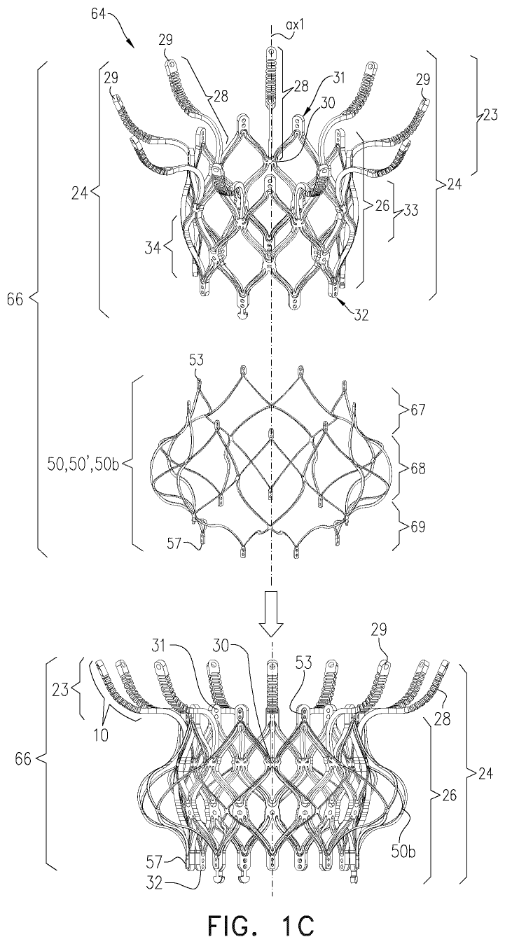

Reference is now made to FIGS. 1A-C, which are schematic illustrations of respective systems 20, 60, and 64 for use at a calcified native heart valve of a patient and comprising respective prosthetic valves 22, 62, and 66, in accordance with some applications of the invention. Each of valves 22, 62, and 66 are deliverable to a heart of the patient typically through a catheter. For some applications, valves 22, 62, and 66 may be delivered using a transcatheter approach, e.g., a transfemoral approach. For some applications, valves 22, 62, and 66 may be delivered using minimally-invasive or surgical techniques. Each of valves 22, 62, and 66 comprises a frame 24 that comprises a valve body 26 that circumscribes a central longitudinal axis ax1 of each of prosthetic valves 22, 62, and 66 and defines a lumen along axis ax1. Frame 24 comprises a plurality of arms 28 (e.g., 3-24 arms, such as 8-12 arms, such as 9 arms) that are attached to and extend from respective circumferential sites 30 of valve body 26 in an upstream direction. Each of arms 28 are configured to extend radially outward to a respective arm-tip in an expanded state of frame 24. The plurality of arms 28 collectively define an annular element 23, or upstream support portion, of the frame that is configured to rest against tissue of the native valve at the atrial surface of the valve. For some applications, arms 28 define atrial anchoring arms. Valves 22, 62, and 66 comprise a plurality of prosthetic leaflets (not shown in FIGS. 1A-C) that are disposed within the lumen of valve body 26. Valves 22, 62, and 66 each comprise a shape-conforming element 40 surrounding the valve body. Shape-conforming element 40 is configured to conform to calcified areas of cardiac tissue of the calcified native heart valve and function as a cushion. That is, portions of shape-conforming element 40 are deformable to accommodate the tissue at the native valve. Some portions of shape-conforming element 40 are deformed by being radially compressed toward a central longitudinal axis ax1 of prosthetic valve 22, 62, or 66 so as to conform to radially-inwardly protruding calcified tissue. Some portions of shape-conforming element 40 expand to accommodate gaps created between abnormal tissue and prosthetic valve 22, 62, or 66. For some applications, shape-conforming element 40 comprises a foam, a sponge, a polymer, and/or a fabric. For some applications, shape-conforming element 40 comprises metal. For some applications, metal such as nitinol and/or stainless steel may be incorporated within shape-conforming element 40. For example, shape-conforming element 40 comprises a foam, a fabric, or a sponge, a metal is reinforced within the foam or sponge.

Typically, shape-conforming element 40 is configured to have a sponge, spring, quality such that element 40 yields to or gives to physical force applied by calcified tissue against valves 22, 62, and 66. In such a manner, shape-conforming element 40 is able to conform and adapt to the topography of the calcified valve, which is typically an asymmetrical and abnormal topography. As such, shape-conforming element 40 facilitates anchoring of prosthetic valve 22 or 62 to calcified tissue of the valve and/or to apparatus (e.g., an annuloplasty structure or a prosthetic valve) already implanted at the malfunctioning or defective native valve.

Typically, shape-conforming element 40 comprises a sponge or a foam, e.g., a shape memory polymer (SMP) foam. For applications in which shape-conforming element 40 comprises a foam or a sponge, shape-conforming element 40 has a pore size of 0.2-3.0 mm. For applications in which shape-conforming element 40 comprises a foam or a sponge, shape-conforming element 40 has a density of 0.01-0.06 g/cm\hat{ }3. For applications in which shape-conforming element 40 comprises a foam or a sponge, shape-conforming element 40 has an ultimate tensile strength of 50-250 kPa. For applications in which shape-conforming element 40 comprises a foam or a sponge, shape-conforming element 40 has a strain at break of 20-80%. For applications in which shape-conforming element 40 comprises a shape-memory foam, shape-conforming element 40 has a glass transition temperature range occurring (i) when element 40 is wet, between 20-40 degrees C. and, (ii) when element 40 is dry, between 50-70 degrees C.

Typically, shape-conforming element 40 is configured to enhance friction and/or provide for better geometrical adapting between prosthetic valves 22, 62, and 66 and the calcified native heart valve. Shape-conforming element 40 is configured to minimize or eliminate paravalvular leakage which could otherwise occur between gap areas created by areas of calcified tissue between the calcified tissue and the prosthetic valve. Since shape-conforming element 40 conforms to asymmetrical tissue formations and/or to lumps by calcified tissue. For applications in which shape-conforming element 40 comprises a foam, shape-conforming element 40 comprises polyurethane. For applications in which shape-conforming element 40 comprises polytetrafluoroethylene, expanded polytetrafluoroethylene. For applications in which shape-conforming element 40 comprises a fabric, shape-conforming element 40 comprises polyethylene terephthalate (commonly known as DACRON™), polyester, and/or any suitable fabric. For applications in which shape-conforming element 40 comprises a fabric, shape-conforming element may comprise a velour fabric, any suitable biocompatible fabric, and/or a combination of fabrics, and/or a combination of fabrics and foam/sponge. Typically, shape-conforming element 40 is not inflatable by a fluid, e.g., shape-conforming element 40 is not mechanically inflatable, although shape-conforming element 40 is passively expandable in the presence of blood. For example, for applications in which shape-conforming element 40 comprises a porous material 42, as shown in FIGS. 1A-B, blood fills the pores of porous material 42. For some applications, shape-conforming element 40 comprises metal such as nitinol and/or stainless steel. For some applications, metal such as nitinol and/or stainless steel may be incorporated within shape-conforming element 40. For example, shape-conforming element 40 comprises a foam or a sponge, a metal is reinforced within the foam or sponge.

For some applications of the present invention, shape-conforming element 40 comprises or is covered with an anti-thrombotic agent. For some applications of the present invention, shape-conforming element 40 comprises or is covered with a fibrosis-enhancing agent.

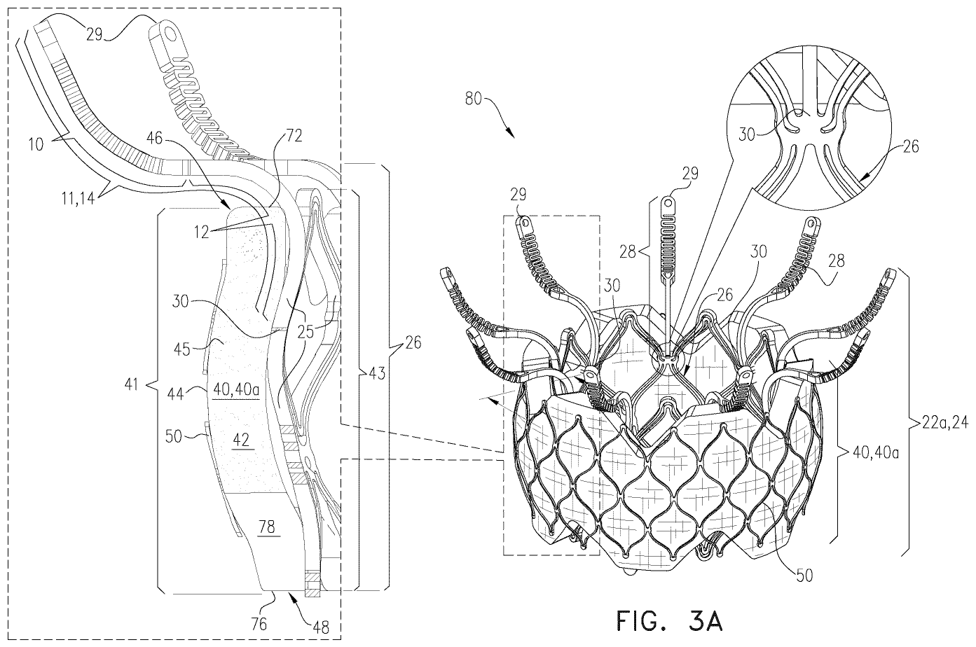

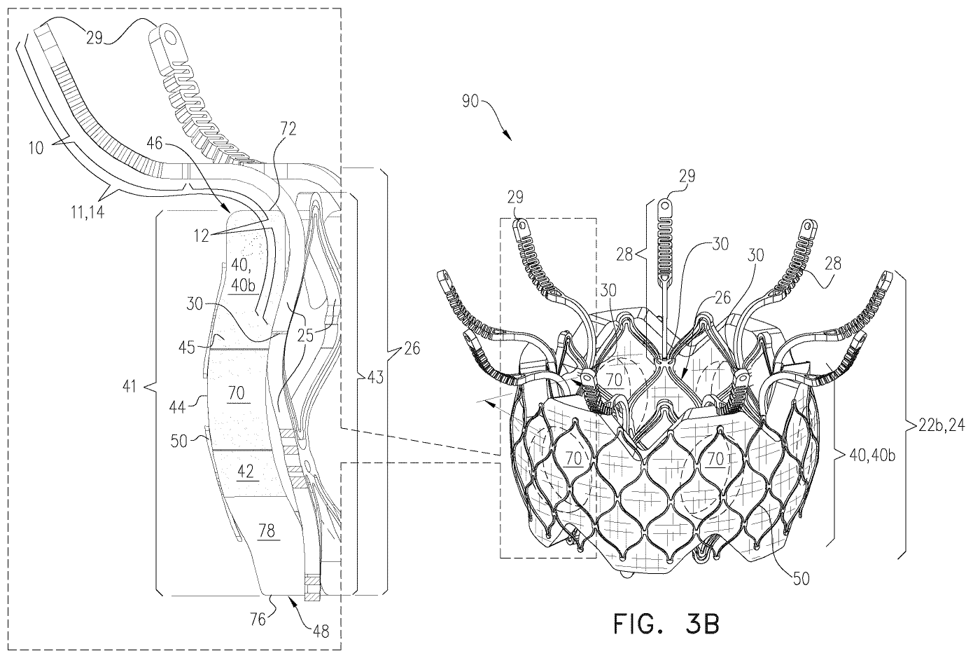

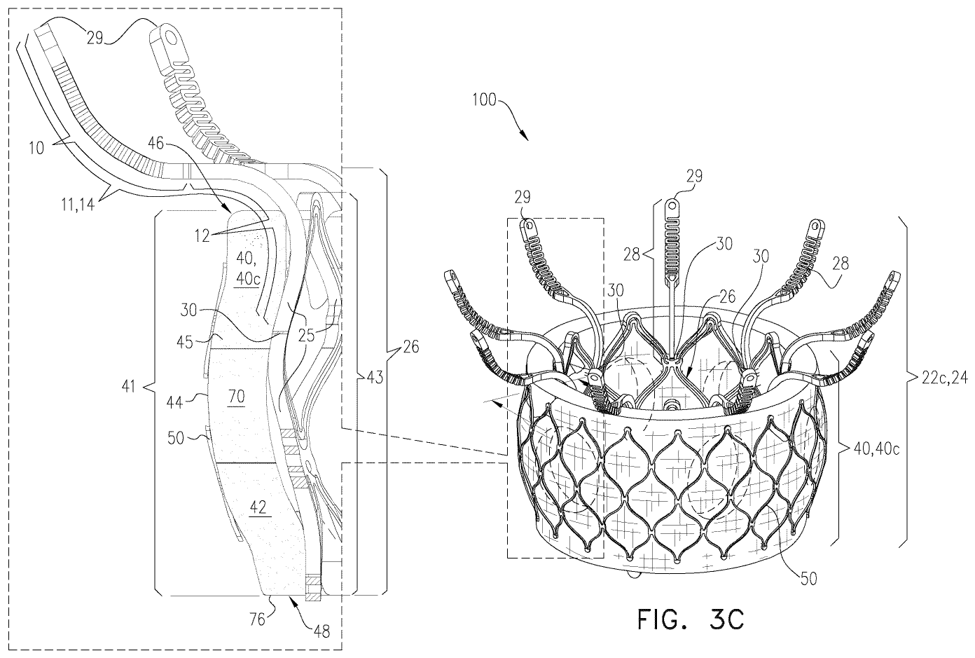

Shape-conforming element 40 is configured to enhance the adaptability of prosthetic valves 22, 62, and 66 to the geometry and topography of the calcified native valve and/or to pre-implanted apparatus at the defective or malfunctioning native valve. Shape-conforming element 40 defines an outer wall having radially inward lateral sections 43 and radially outward lateral sections 41 (labeled in FIGS. 3A-C, 5A, and 7-8). When conforming to the shape of the calcified valve and/or to apparatus already implanted at the native valve, radially outward lateral section 41 can move toward radially inward lateral section 43 by 20-90%, e.g., by 60-70%, from a resting state of sections 41 and 43. Typically, shape-conforming element 40 undergoes a radial deformation of between 1 and 9 mm, e.g., 5 mm, during implantation. As shown in FIGS. 1A-B, 2A-9B, and 11, the outer wall of shape-conforming element 40 (1) comprises material, e.g., foam, sponge, or fabric and (2) surrounds material (e.g., foam, sponge, or fabric) or empty space. For some applications of the present invention, shape-conforming element 40 comprises material, e.g., foam, sponge, or fabric and is hollow. For some applications of the present invention, shape-conforming element 40 comprises material, e.g., foam, sponge, or fabric and also metal. For some applications of the present invention, shape-conforming element 40 comprises a braided mesh, woven mesh, or stent structure and is hollow, as is shown in FIG. 7.

Typically, radially outward lateral section 41 is mechanically isolated from radially inward lateral section 43 such that during deformation of shape-conforming element 40, radially outward laterally section 41 changes shape while radially inward lateral section 43 remains undeformed.

Shape-conforming element 40 has a first cross-sectional shape in a resting state of shape-conforming element 40 and is deformable into a second cross-sectional shape to adapt to a shape of native tissue at the native valve or to apparatus implanted at tissue at the native heart valve, in a deployed state of prosthetic valve 22, 62, or 66. Shape-conforming element 40 is mechanically isolated from frame 24 such that frame 24 maintains an undeformed shape when shape-conforming element 40 has deformed into the second cross-sectional shape.

For applications in which shape-conforming element 40 comprises porous material 42, e.g., a foam or a sponge, shape-conforming element 40 is surrounded by a covering 44, which may include a flexible sheet, such as a fabric, e.g., including polyester. For some applications, covering 44 comprises a braided mesh. Covering 44 is configured to prevent migration of pieces of shape-confirming element 40 away from valve body 26. Typically, but not necessarily, covering 44 comprises polyethylene terephthalate (commonly known as DACRON™).

Typically, the outer surface of shape conforming element 40 and/or of covering 44 is smooth. That is, the purpose of shape-conforming element 40 is to increase surface area contact with abnormal tissue by conforming to the tissue. Such deformation increases surface area contact and friction between the prosthetic valves described herein and tissue of the native valve. It is hypothesized that the deformation of shape-conforming element 40 is what helps anchor, properly align, and seal the prosthetic valves described herein to the native valve and/or to apparatus already implanted at the native valve. For applications in which shape-conforming element 40 comprises fabric, the fabric is typically smooth and is woven so as to minimize or eliminate protruding loops of thread or radially-extending tips, which are typically provided in terrycloth or other toweling materials.

Prosthetic valves 22, 62, and 66 are each typically configured to be implanted at a calcified atrioventricular valve, e.g., a mitral valve or a tricuspid valve. The scope of the present invention includes prosthetic valves 22, 62, and 66 being configured to be implanted at the aortic valve (e.g., to treat aortic stenosis) or at the pulmonary valve.

For some applications of the present invention valves 22, 62, and 66 are self-expandable and comprise a shape-memory material, e.g., nitinol. For some applications of the present invention valves 22, 62, and 66 are balloon-expandable and comprise a metal, e.g., stainless steel or cobalt chrome. For some applications of the present invention valves 22, 62, and 66 have respective portions that are self-expandable and balloon-expandable. For example, valve body 26 may be balloon-expandable and arms 28 are self-expandable.

Valve body 26 defines an upstream end 31 and a downstream end 32. Valve body 26 comprises a plurality of struts 25 that are arranged such that body 26 assumes a generally cylindrical shape, e.g., a bulging cylinder having a barrel shape, as shown, or a straight cylinder (not shown). For some applications of the present invention, valve body 26 is shaped as an hourglass. For some applications of the present invention, valve body 26 is shaped so as to define concave portions, e.g., such that a transverse cross-section of body 26 at an intermediate portion of body 26 that is between ends 31 and 32 and measured along an axis that is perpendicular to axis ax1, is narrower than the portions of valve body 26 at upstream and downstream ends 31 and 32. Typically, adjacent ascending and descending struts 25 along an upstream perimeter of valve body 26 form peaks at upstream end 31 and valleys that are downstream of upstream end 31. These valleys define respective circumferential sites 30 that are at an intermediate location between upstream end 31 and downstream end 32 of valve body 26. Typically, adjacent ascending and descending struts 25 at a downstream perimeter of valve body 26 form valleys at downstream end 32 and peaks that are upstream of downstream end 32.

Struts 25 are typically arranged in one or more rings, e.g., a first (e.g., upstream) ring 33 and a second (e.g., downstream) ring 34. For some applications, and as shown, valve body 26 comprises exactly two rings, as shown. Each ring is defined by a pattern of alternating peaks and troughs, the peaks being further upstream than the troughs. Valve body 26 is defined by a repeating pattern of cells that extends around central longitudinal axis ax1. In some embodiments, and as shown, valve body 26 is defined by two stacked, tessellating rings of cells. In the expanded state valve body 26, these cells may be narrower at their upstream and downstream extremities than midway between these extremities. For example, and as shown, the cells may be roughly diamond, hexagonal, or asteroid in shape. Each arm 28 is attached at its downstream end to and extends from a respective circumferential site 30 that is at the connection between two adjacent cells of upstream ring 33 (alternatively described as being at the upstream extremity of cells of downstream ring 34). The configuration of frame 24 is such that it provides strong and robust radial and oval forces.

It is hypothesized by the inventors that this lower position of arms 28, while maintaining the length of the lumen of valve body 26, advantageously reduces the distance that valve body 26 (i.e., downstream end 32 thereof) extends into the ventricle of the patient, and thereby reduces a likelihood of inhibiting blood flow out of the ventricle through the left ventricular outflow tract. It is further hypothesized that this position of arms 28 reduces radial compression of the inner frame tubular portion by movement of the heart, due to greater rigidity of valve body 26 at sites 30 (which is supported by two adjacent cells).

Typically, but not necessarily, valve body 26 assumes a double-strut pattern in which two struts 25 run alongside each other, as shown in the enlarged image. Struts 25 are connected at nodes 27. This double-strut pattern is hypothesized by the inventors to increase strength and rigidity. In particular, the double-strut pattern is hypothesized by the inventors to increase the resistance of the valve body to compression toward axis ax1, including resistance to circumferential compression (e.g., compression that would otherwise reduce the diameter of valve body 26, but that would retain valve body 26 in a generally cylindrical shape) and localized compression (e.g., compression that would otherwise reduce the diameter of valve body 26 at only certain locations, causing valve body 26 to become more oval in transverse cross-section). It is to be noted that the scope of the present invention includes a valve body 26 that is arranged from single struts.

For some applications, annular element 23 that defines an upstream support portion includes the plurality of arms 28. In an expanded state of frame 24, each arm 28 extends radially outward from valve body 26 (e.g., from circumferential site 30 of valve body 26) to arm terminal end at arm-tip 29. As shown, arms 28 are not interconnected and movement of one arm 28 is not affected by movement of an adjacent arm 28. As shown in the enlarged image of arm 28, each arm 28, when extending radially outward from valve body 26, includes a radially-extending portion 11 having a downstream surface 14 having a downstream-directed convex portion 10 adjacent arm-tip 29. In the expanded state of frame 24, each arm 28 has (1) a downstream-directed concave portion 12 at a radially-inner portion of arm 28, and (2) downstream-directed convex portion 10 that is between downstream-directed concave portion 12 and arm-tip 29. Typically, downstream-directed convex portion 10 and downstream-directed concave portion 12 are angled relative to each other. Arms 28 are typically flexible, as shown, an comprise a spring arranged in an “S”-shape, at least in part, e.g., at downstream-directed convex portion 10. With such flexibility, arms 28, in particular downstream-directed convex portions 10 are flexible, soft, have a spring quality, and self-conform to the atrial morphology. For some applications, arms 28 are coupled to valve body 26 such that each arm 28 may deflect independently of adjacent arms during implantation (e.g., due to anatomical topography).

Typically, valves 22, 62, and 66 are each covered by a fabric covering (not shown) which may comprise a single, unitary sheet of fabric or a plurality of sheets stitched together. For some arms 28 are covered with a covering, e.g., extending between arms 28 to form an annular shape. It is hypothesized that this reduces a likelihood of paravalvular leakage. For such embodiments, excess covering may be provided between arms 28 of the upstream support portion, so as to facilitate their independent movement. The covering can cover the atrial, upstream side of the upstream support portion (i.e., the plurality of arms 28), and may additionally or alternatively cover the ventricular, downstream side of the upstream support portion. For example, the covering may extend over tips 29 of arms 28 and down the outside of the arms, or a separate piece of covering may be provided on the ventricular, downstream side of the upstream support portion.

Alternatively, each arm 28 may be individually covered in a sleeve of covering, thereby facilitating independent movement of arms 28.

For some applications, as shown in FIG. 1A, valve 22 comprises an outer flexible structure 50 comprising an expandable element, e.g., a stent structure, that surrounds shape-conforming element 40. Outer flexible structure 50 is configured to enhance friction between prosthetic valve 22 and the calcified native heart valve. Outer flexible structure 50 defines an upstream end and a downstream end. Outer flexible structure 50 comprises a plurality of struts that are arranged such that outer flexible structure 50 assumes a generally cylindrical outer flexible structure 50a, e.g., a bulging cylinder having a barrel shape, as shown, or a straight cylinder (not shown). For some applications of the present invention, outer flexible structure 50 undergoes a radial deformation of between 1 and 9 mm, e.g., 5 mm, during implantation.

For some applications of the present invention, outer flexible structure 50 is shaped as an hourglass. For some applications of the present invention, outer flexible structure 50 is shaped so as to define concave portions, e.g., such that a transverse cross-section of outer flexible structure 50 at an intermediate portion of outer flexible structure 50 that is between the upstream and downstream ends and measured along an axis that is perpendicular to axis ax1, is narrower than the portions of outer flexible structure 50 at the upstream and downstream ends. Typically, adjacent ascending and descending struts along an upstream perimeter of outer flexible structure 50 form peaks at the upstream end and valleys that are downstream of the upstream end. Typically, adjacent ascending and descending struts at a downstream perimeter of outer flexible structure 50 form valleys at the downstream end and peaks that are upstream of the downstream end.

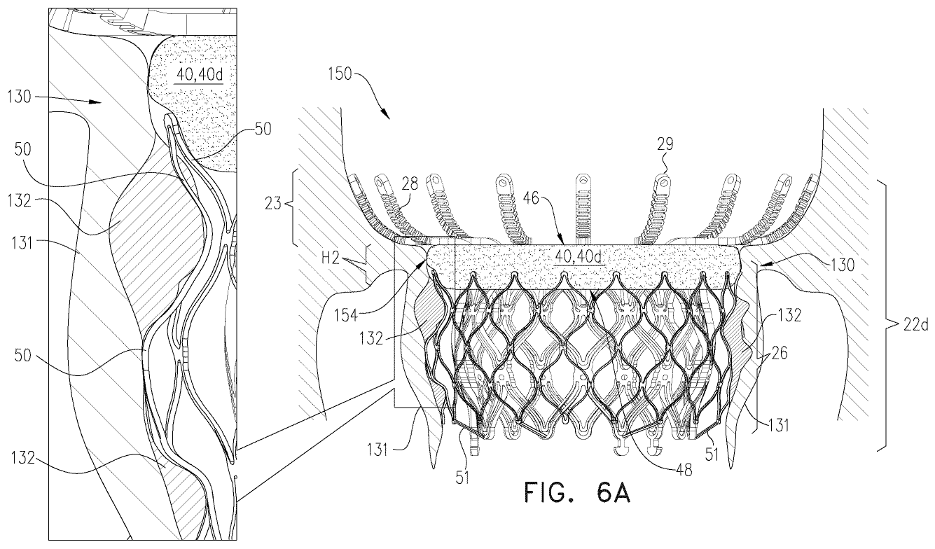

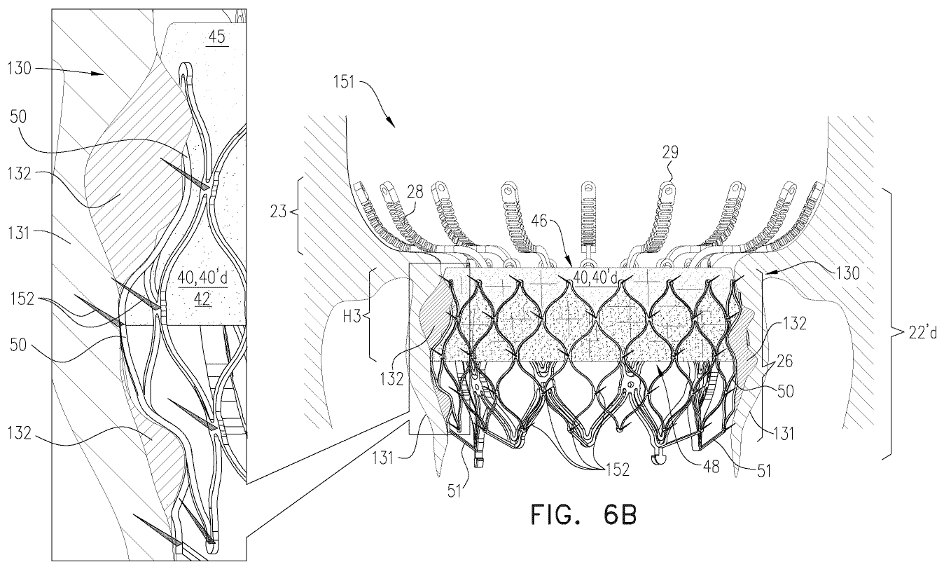

For some applications, outer flexible structure 50 includes a plurality of anchors (e.g., barbs, teeth, or hooks) (not shown but, for example, anchors 152 shown hereinbelow with reference to FIG. 6B) that extend and protrude out of a ventricular-facing surface of structure 50. The anchors press into tissue of the calcified native valve thereby inhibiting movement of prosthetic valve 22 (in addition to inhibition of downstream movement provided by the geometry of the upstream support portion of annular element 23). For some applications, the anchors may be concentrated on structure 50 or may be sparsely positioned on structure 50. For some applications, the anchors may protrude radially and disposed circumferentially with respect to valve body 26 such that tissue of the native valve is captured between the anchors and the prosthetic valve by rotating the prosthetic valve about axis ax1.

Typically, outer flexible structure 50 is coupled to shape-conforming element 40 with radial strength and/or is sutured to shape-conforming element 40. For some applications, the upstream ends of outer flexible structure 50 are not fixedly coupled to either shape-conforming element 40 or to upstream end 32 of valve body 26 of frame 24. In such applications, the upstream ends of outer flexible structure 50 slide longitudinally with respect to upstream end 32 of valve body 26 of frame 24 and/or to the upstream end of shape-conforming element 40 during expanding and contracting of the prosthetic valve.

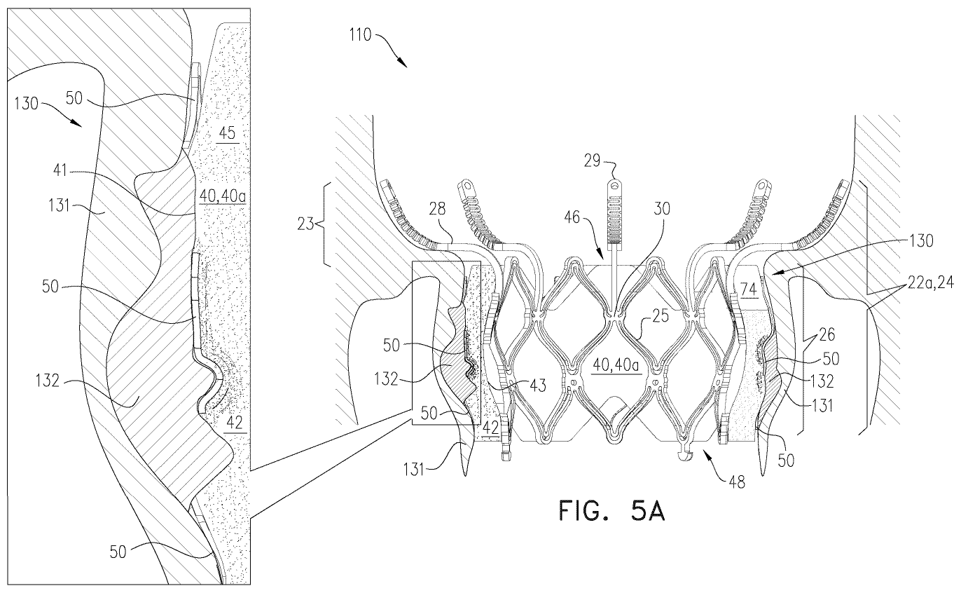

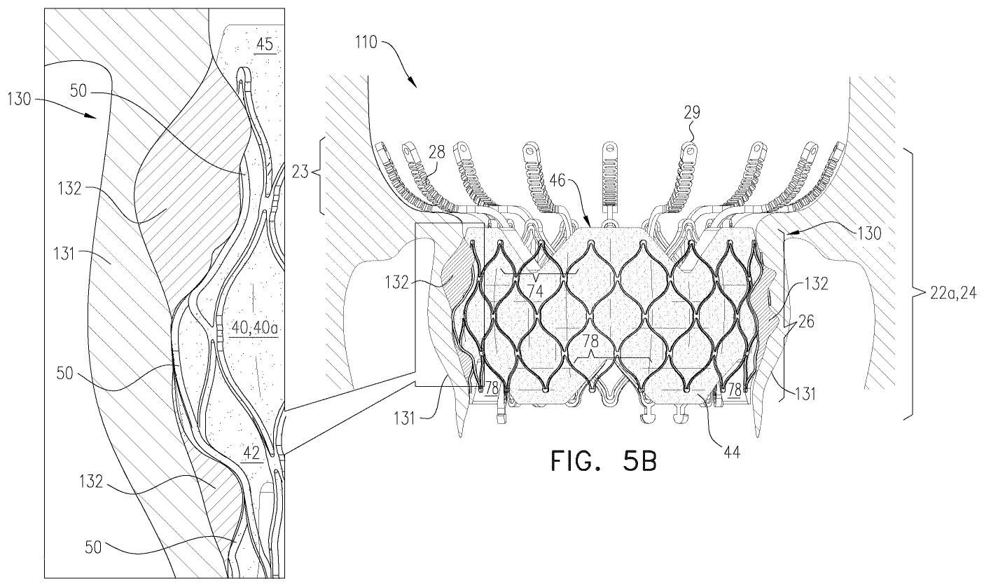

For some applications, outer flexible structures 50 and 50a comprises a metal deformation element 50′ (e.g., as shown hereinbelow with reference to FIGS. 5A-B, 6A-B, and 7-11) which is configured to conform to tissue or to pre-implanted apparatus. That is, portions of outer flexible structure 50 are deformable to accommodate the tissue at the native valve or pre-implanted apparatus at the native valve. Some portions of outer flexible structure 50 are deformed by being radially compressed toward central longitudinal axis ax1 of prosthetic valve 22 so as to conform to radially-inwardly protruding calcified tissue or pre-implanted apparatus. For some applications, outer flexible structure 50 comprises metal such as nitinol and/or stainless steel. In some applications, metal deformation element 50′ is sufficient to conform to the tissue or to pre-implanted apparatus such that shape-conforming element 40 is absent from prosthetic valve 22 shown in FIG. 1A (e.g., similarly to prosthetic valve 66 described hereinbelow with reference to FIG. 1C).

For some applications of the present invention, the purpose of metal deformation element 50′ is to increase surface area contact with abnormal tissue by conforming to the tissue. Such deformation increases surface area contact and friction between the prosthetic valves described herein and tissue of the native valve. It is hypothesized that the deformation of metal deformation element 50′ is what helps anchor, properly align, and seal the prosthetic valves described herein to the native valve and/or to apparatus already implanted at the native valve.

For some applications, outer flexible structure 50 includes a plurality of anchors (e.g., barbs, teeth, or hooks) (not shown but, for example, anchors 152 shown hereinbelow with reference to FIG. 6B) that extend and protrude out of a ventricular-facing surface of structure 50. The anchors press into tissue of the calcified native valve thereby inhibiting movement of prosthetic valve 22 (in addition to inhibition of downstream movement provided by the geometry of the upstream support portion of annular element 23). For some applications, the anchors may be concentrated on structure 50 or may be sparsely positioned on structure 50. For some applications, the anchors may protrude radially and disposed circumferentially with respect to valve body 26 such that tissue of the native valve is captured between the anchors and the prosthetic valve by rotating the prosthetic valve about axis ax1.

Typically, for applications in which valves 22, 62, and 66 are self-expandable, outer flexible structure 50 comprises a self-expanding frame and comprises a shape memory material (e.g., nitinol). For applications in which valves 22, 62, and 66 are balloon-expandable (e.g., valve body 26 is balloon-expandable) outer flexible structure 50 comprise a balloon-expandable metal, e.g., stainless steel or cobalt chrome, or a self-expanding metal comprising a shape memory material (e.g., nitinol).

For some applications, outer flexible structure 50 comprises a plurality of struts. As shown, for some applications, outer flexible structure 50 comprises a unitary structure shaped so as to define three rows of tessellating cells. Structure 50 may be cylindrical and comprise a tubular structure. Structure 50 may be generally cylindrical and shaped as a barrel, as shown. For some application, outer flexible structure 50 comprises two discrete rows of cells arranged in a single upstream ring separate from a single downstream ring (configuration not shown). For some applications, outer flexible structure 50 comprises a braided mesh.

For some applications, as shown in FIG. 1B, prosthetic valve 62 does not comprise expandable outer flexible structure 50.

FIG. 1C shows prosthetic valve 66 without a shape-conforming element 40 between valve body 26 of frame 24 and outer flexible structure 50. Additionally, as shown, outer flexible structure 50 comprises a bulging outer flexible structure 50b having an upstream narrow portion 67, a downstream narrow portion 69 and an intermediate wide portion 68. For some applications, outer flexible structure 50b does not comprise upstream narrow portion 67. For some applications, as shown, intermediate wide portion 68 is curved radially outward such that portion 68 defines a cylinder, a ring or a toroid. Intermediate wide portion 68 typically does not contact an outer surface of valve body 26. When conforming to the shape of the calcified valve and/or to apparatus already implanted at the native valve, intermediate wide portion 68 can move toward the outer surface of valve body 26 by being 20-90%, e.g., by 60-70%, radially compressed from a resting state portion 68. Typically, intermediate wide portion 68 undergoes a radial deformation of between 1 and 9 mm, e.g., 5 mm, during implantation.

For some applications, valve 66 does not comprise bulging outer flexible structure 50b, but rather may comprise outer flexible structure generally cylindrical outer flexible structure 50a as shown in FIG. 1A.

Outer flexible structure 50b defines an upstream end 53 and a downstream end 57. Outer flexible structure 50b comprises a plurality of struts that are arranged such that outer flexible structure 50b a bulging cylinder having a barrel shape, as shown. For some applications of the present invention, outer flexible structure 50b undergoes a radial deformation of between 1 and 9 mm, e.g., 5 mm, during implantation.

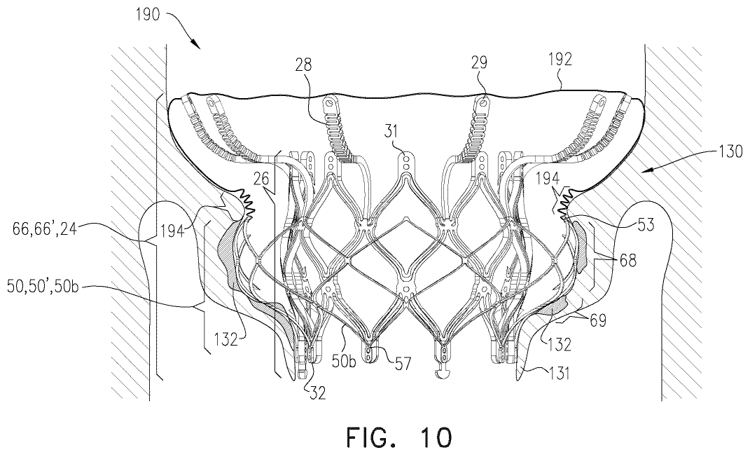

Typically, outer flexible structure 50b comprises a metal deformation element 50′ (e.g., as shown hereinbelow with reference to FIG. 10) which is configured to conform to tissue or to pre-implanted apparatus. That is, portions of outer flexible structure 50b are deformable to accommodate the tissue at the native valve or pre-implanted apparatus at the native valve. Some portions of outer flexible structure 50b are deformed by being radially compressed toward central longitudinal axis ax1 of prosthetic valve 66 so as to conform to radially-inwardly protruding calcified tissue or pre-implanted apparatus. For some applications, outer flexible structure 50b comprises metal such as nitinol and/or stainless steel.

Typically, intermediate wide portion 68 outer flexible structure 50b is mechanically isolated from valve body 26 of frame 24 such that during deformation of intermediate portion 68, such that intermediate portion 68 functions as metal deformation element 50′, intermediate portion 68 changes shape while valve body 26 remains undeformed at least at the longitudinal section of body 26 adjacent intermediate portion 68. For some applications, as shown, intermediate wide portion 68 is curved radially outward such that portion 68 defines a cylinder, a ring or a toroid.

As shown in FIG. 1C, outer flexible structure 50b surrounds valve body 26 of frame 24. Downstream ends 57 of flexible structure 50b may be fixedly coupled to downstream end 32 of valve body 26 of frame 24. Downstream ends 57 of flexible structure 50b may be fixedly coupled to downstream end 32 of valve body 26 of frame 24 (1) by struts creating a space between downstream ends 57 of structure 50b and downstream end 32 of valve body 26 of frame 24, (2) by being directly coupled to valve body 26 of frame 24 (as shown) such as by welding, thereby eliminating a space between downstream ends 57 of structure 50b and downstream end 32 of valve body 26 of frame 24, or (3) by being coupled to valve body 26 of frame 24 using sutures or other coupling elements.

For some applications, upstream ends 53 of flexible structure 50b may be fixedly coupled to upstream end 32 of valve body 26 of frame 24 (1) by struts creating a space between upstream ends 53 of structure 50b and upstream end 31 of valve body 26 of frame 24, (2) by being directly coupled to valve body 26 of frame 24 (as shown) such as by welding, thereby eliminating a space between upstream ends 53 of structure 50b and upstream end 31 of valve body 26 of frame 24, (3) by being coupled to valve body 26 of frame 24 using sutures or other coupling elements, or (4) by being coupled to valve body 26 of frame 24 using fabric, as described hereinbelow with reference to FIG. 10.

For some applications, upstream ends 53 of flexible structure 50b are not fixedly coupled to upstream end 32 of valve body 26 of frame 24. In such applications, upstream ends 53 slide longitudinally with respect to upstream end 32 of valve body 26 of frame 24 during expanding and contracting of the prosthetic valve.

For some applications, outer flexible structure 50b includes a plurality of anchors (e.g., barbs, teeth, or hooks) (not shown but, for example, anchors 152 shown hereinbelow with reference to FIG. 6B) that extend and protrude out of a ventricular-facing surface of structure 50b. The anchors press into tissue of the calcified native valve thereby inhibiting movement of prosthetic valve 66 (in addition to inhibition of downstream movement provided by the geometry of the upstream support portion of annular element 23). For some applications, the anchors may be concentrated on structure 50b or may be sparsely positioned on structure 50b. For some applications, the anchors may protrude radially and disposed circumferentially with respect to valve body 26 such that tissue of the native valve is captured between the anchors and the prosthetic valve by rotating the prosthetic valve about axis ax1.

For some applications as shown in FIG. 1C, upstream ends 53 of outer flexible structure 50b are upstream of circumferential sites 30 at which each arm 28 is attached to and extends from valve body 26. As shown, circumferential sites 30 are longitudinally between upstream ends 53 and downstream ends 57 of outer flexible structure 50b. Additionally, for each arm 28, downstream-directed convex portion 10 is upstream of upstream end 53 of outer flexible structure 50b.

Reference is again made to FIGS. 1A-C. For some applications of the present invention, prosthetic valves 22, 62, and 22′ are couplable to a ring, e.g., an annuloplasty ring such as a full annuloplasty ring and a partial annuloplasty ring, that is implantable or has already previously been implanted at a defective or malfunctioning native valve. For some applications of the present invention, prosthetic valves 22, 62, and 22′ are couplable to a prosthetic valve has already previously been implanted at the defective or malfunctioning native valve. In such cases, the pre-implanted prosthetic valve may comprise a prosthetic valve that is not functioning properly.

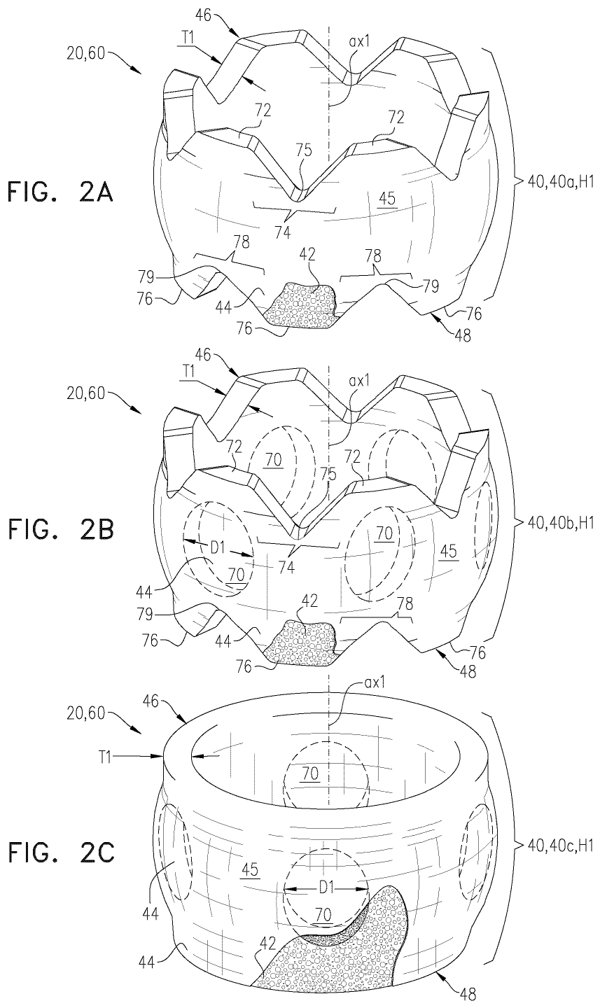

Reference is now made to FIGS. 2A-C, which are schematic illustrations of various shapes of shape-conforming element 40, in accordance with some applications of the invention. Typically, shape-conforming element 40 has wall 45 having a thickness T1 of 3-9 mm, e.g., 3-7 mm, e.g., 5 mm, measured from a radially innermost portion of shape-conforming element 40 to a radially outermost portion of shape-conforming element 40. Typically, shape-conforming element 40 has a uniform thickness along the longitudinal length of element 40. For some applications, when compressed, shape-conforming element can reach a thickness of 1-2 mm, e.g., 1.5 mm. Typically, shape-conforming element 40 has a height H1 of 5-20 mm measured along longitudinal axis ax1 between an upstream surface at an upstream end 46 of shape-conforming element 40 and a downstream surface at a downstream end 48 of shape-conforming element 40.

For some applications, shape-conforming element 40 assumes a generally cylindrical shape, e.g., a bulging cylinder having a barrel shape, as shown, or a straight cylinder (not shown). For some applications of the present invention, shape-conforming element 40 is shaped as an hourglass. For some applications of the present invention, shape-conforming element 40 is shaped so as to define concave portions, e.g., such that a transverse cross-section of element 40 at an intermediate portion of element 40 that is between ends 46 and 48 and measured along an axis that is perpendicular to axis ax1, is narrower than the portions of shape-conforming element 40 at upstream and downstream ends 46 and 48.

In FIG. 2A, shape-conforming element 40 is shaped so as to define an undulating shape-conforming element 40a that has at least one surface (i.e., both surfaces at ends 46 and 48) that is arranged in an undulating pattern (e.g., a wave pattern, ascending and descending pattern, or sinusoidal pattern) around an outer surface of valve body 26 (as shown hereinbelow with reference to FIG. 3A). As shown, the undulating upstream surface at upstream end 46 is rotationally offset with respect to the undulating downstream surface at downstream end 48.

For some applications, undulating shape-conforming element 40a is shaped so as to define at least one surface (i.e., both surfaces at ends 46 and 48) that is arranged in a zig-zag pattern around an outer surface of valve body 26 (as shown hereinbelow with reference to FIG. 3A). For some applications, the zig-zag pattern at the upstream surface at upstream end 46 is rotationally offset with respect to the zig-zag pattern at the downstream surface at downstream end 48.

The undulating shape-conforming element 40a is shaped so as to reduce the overall profile of prosthetic valves 22 and 62 in their compressed state during delivery while maximizing surface area contact between (1) prosthetic valves 22 and 62 in their expanded state and (2) calcified tissue of the calcified native valve during implantation.

As shown in FIG. 2A, upstream end 46 has a plurality of upstream surfaces 72 alternating with a plurality of downstream-directed cutout portions 74. Each downstream-directed cutout portion 74 has respective descending and ascending edges connected at a downstream-directed vertex 75. Downstream end 48 has a plurality of downstream surfaces 76 alternating with a plurality of upstream-directed cutout portions 78. Each upstream-directed cutout portion 78 has respective ascending and descending edges connected at an upstream-directed vertex 79. Downstream-directed vertices 75 are rotationally offset with respect to upstream-directed vertices 79. Each downstream-directed vertex 75 faces a midpoint of an opposite downstream surface 76 of downstream end 48 of undulating shape-conforming element 40a. Each upstream-directed vertex 79 faces a midpoint of an opposite upstream surface 72 of upstream end 46 of undulating shape-conforming element 40a. For some applications, vertices 75 and 79 are pointed. For some applications, vertices 75 and 79 are rounded. For some applications, surfaces 72 and 76 are straight. For some applications, surfaces 72 and 76 are curved. Surfaces 72 are rotationally offset with respect to surfaces 76. For some applications of the present invention, surfaces 72 and 76 and vertices 75 and 79 are not rotationally offset.

In FIG. 2B, shape-conforming element 40 is shaped so as to define an undulating, windowed shape-conforming element 40b that has at least one surface (i.e., both surfaces at ends 46 and 48) that is arranged in an undulating pattern (e.g., a wave pattern, ascending and descending pattern, or sinusoidal pattern) around an outer surface of valve body 26 (as shown hereinbelow with reference to FIG. 3B). As shown, the undulating upstream surface at upstream end 46 is rotationally offset with respect to the undulating downstream surface at downstream end 48.

Undulating, windowed shape-conforming element 40b is shaped so as to define a plurality of windows 70 through wall 45 of shape-conforming element 40. Each window 70 has a longest dimension D1 of 2-7 mm in the expanded state of frame 24 of prosthetic valves 22 and 62. Shape-conforming element 40b is shaped so as to define 3-18 windows 70, e.g., 6 windows 70, through wall 45. Windows 70 are configured to reduce the overall profile of prosthetic valves 22 and 62 in their compressed state during delivery while maximizing surface area contact between (1) prosthetic valves 22 and 62 in their expanded state and (2) calcified tissue of the calcified native valve during implantation. As described hereinabove, shape-conforming element 40 is surrounded by a covering 44. Covering 44 also covers windows 70, as shown.

For some applications, undulating, windowed shape-conforming element 40b is shaped so as to define at least one surface (i.e., both surfaces at ends 46 and 48) that is arranged in a zig-zag pattern around an outer surface of valve body 26 (as shown hereinbelow with reference to FIG. 3B). For some applications, the zig-zag pattern at the upstream surface at upstream end 46 is rotationally offset with respect to the zig-zag pattern at the downstream surface at downstream end 48.

Undulating, windowed shape-conforming element 40b is shaped so as to reduce the overall profile of prosthetic valves 22 and 62 in their compressed state during delivery while maximizing surface area contact between (1) prosthetic valves 22 and 62 in their expanded state and (2) calcified tissue of the calcified native valve during implantation.

As shown in FIG. 2B, upstream end 46 has a plurality of upstream surfaces 72 alternating with a plurality of downstream-directed cutout portions 74. Each downstream-directed cutout portion 74 has respective descending and ascending edges connected at a downstream-directed vertex 75. Downstream end 48 has a plurality of downstream surfaces 76 alternating with a plurality of upstream-directed cutout portions 78. Each upstream-directed cutout portion 78 has respective ascending and descending edges connected at an upstream-directed vertex 79. Downstream-directed vertices 75 are rotationally offset with respect to upstream-directed vertices 79. Each downstream-directed vertex 75 faces a midpoint of an opposite downstream surface 76 of downstream end 48 of undulating, windowed shape-conforming element 40b. Each upstream-directed vertex 79 faces a midpoint of an opposite upstream surface 72 of upstream end 46 of undulating, windowed shape-conforming element 40b. For some applications, vertices 75 and 79 are pointed. For some applications, vertices 75 and 79 are rounded. For some applications, surfaces 72 and 76 are straight. For some applications, surfaces 72 and 76 are curved. Surfaces 72 are rotationally offset with respect to surfaces 76. For some applications of the present invention, surfaces 72 and 76 and vertices 75 and 79 are not rotationally offset.

FIG. 2C shows shape-conforming element 40 comprising a windowed shape-conforming element 40c with similar properties to those described hereinabove with reference to FIG. 2B, with the exception that ends 46 and 48 are not undulating, and windowed shape-conforming element 40c is not arranged in an undulating or zig-zag pattern around the outer surface of valve body 26, as shown hereinbelow with reference to FIG. 3C. Rather, the upstream surface at upstream end 46 of shape-conforming element 40c forms a complete ring, and the downstream surface at downstream end 48 of shape-conforming element 40c forms a complete ring.

Windowed shape-conforming element 40c is shaped so as to reduce the overall profile of prosthetic valves 22 and 62 in their compressed state during delivery while maximizing surface area contact between (1) prosthetic valves 22 and 62 in their expanded state and (2) calcified tissue of the calcified native valve during implantation.

Reference is now made to FIGS. 1A-B and 2A-C. It is to be noted that although undulating shape-conforming element 40a is shown in FIGS. 1A-B, any one of undulating, windowed shape-conforming element 40b, windowed shape-conforming element 40c, or any shape-conforming element 40 having any suitable shape may be used in combination with prosthetic valves 22 and 62. It is to be noted that for clarity of illustration, a cutaway in covering 44 is shown to show underlying porous material 42.

FIGS. 3A-C are schematic illustrations of systems 80, 90, and 100 showing different prosthetic valves 22a, 22b, and 22c comprising the respective shape-conforming elements 40a, 40b, and 40c of FIGS. 2A-C, in accordance with some applications of the invention. It is to be noted that system 80 is similar to system 20 described hereinabove. It is to be noted that although FIGS. 3A-C show the respective shape-conforming elements 40 part of prosthetic valve 22 of FIG. 1A, the shape-conforming elements 40 are also part of prosthetic valve 62 of FIG. 1B. FIGS. 3A-C show enlarged cross-sections sections of each of valves 22a, 22b, and 22c. As shown, for each prosthetic valve, upstream end 46 of shape-conforming element 40 is upstream of circumferential sites 30 at which each arm 28 is attached to and extends from valve body 26. As shown, circumferential sites 30 are longitudinally between upstream end 46 and downstream end 48 of shape-conforming element 40. Additionally, for each arm 28, downstream-directed convex portion 10 is upstream of upstream end 46 of shape-conforming element 40. Typically, prosthetic valves 22a, 22b, and 22c have a ventricular portion having a longest dimension measured perpendicularly to axis ax1 that is around 30-40 mm, e.g., 35 mm. As mentioned above, shape-conforming element has a thickness T1 of about 5 mm such that valve body 26 has a longest dimension measured perpendicularly to axis ax1 that is around 25 mm.

From circumferential site 30 at which each arm 28 is connected to valve body 26, arm extends radially outward from site 30, in an upstream direction forming radially-extending portion 11 which (1) curves to form downstream-directed concave portion 12 at a radially-inner portion of arm 28, (2) further curves at a point further radially-outward from the radially-inward portion of arm 28 and adjacent downstream-directed concave portion 12 to form downstream-directed convex portion 10, and (3) terminates at arm-tip 29.

Each of shape-conforming elements 40 shown in FIGS. 3A-C covers between 15-95% of valve body 26, depending on whether shape-conforming element is (1) undulating as shown in undulating shape-conforming element 40a (FIGS. 2A and 3A) (2) undulating, windowed shape-conforming element 40b (FIGS. 2B and 3B), or (3) windowed shape-conforming element 40c (FIGS. 2C and 3C).

The cross-section image in FIG. 3A shows upstream-directed cutout portion 78 in wall 45 of undulating, shape-conforming element 40a. The cross-section image in FIG. 3B shows upstream-directed cutout portion 78 and window 70 in wall 45 of undulating, windowed shape-conforming element 40b. The cross-section image in FIG. 3C window 70 in wall 45 of windowed shape-conforming element 40c.

Reference is now made to FIGS. 1B, 2A-C, and 3A-C. It is to be noted that although prosthetic valve 62 of FIG. 1B is shown comprising undulating shape-conforming element 40a described in detail in FIGS. 2A and 3A, prosthetic valve 62 may also comprise undulating, windowed, shape-conforming element 40b described in detail in FIGS. 2B and 3B, or windowed shape-conforming element 40c described in detail in FIGS. 2C and 3C.

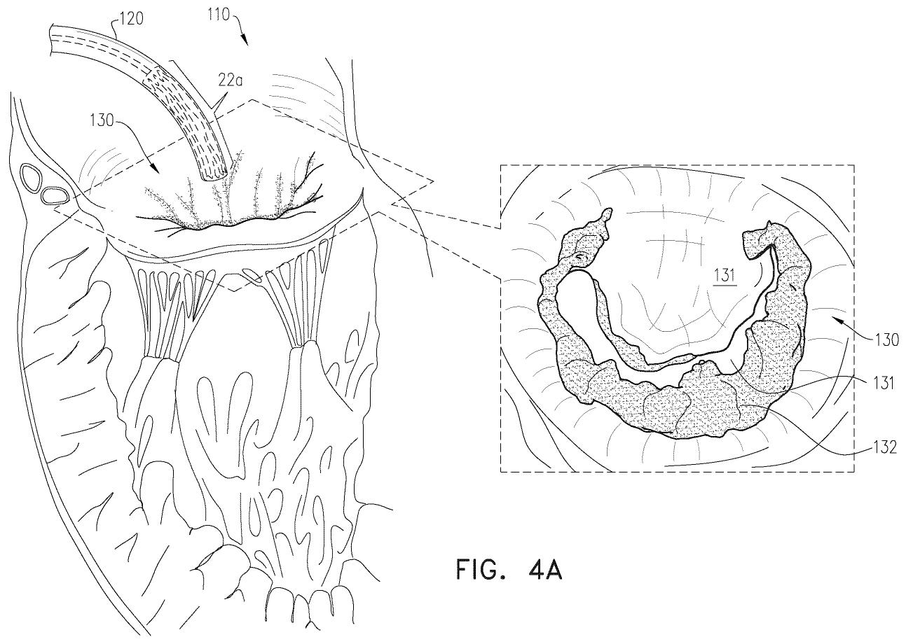

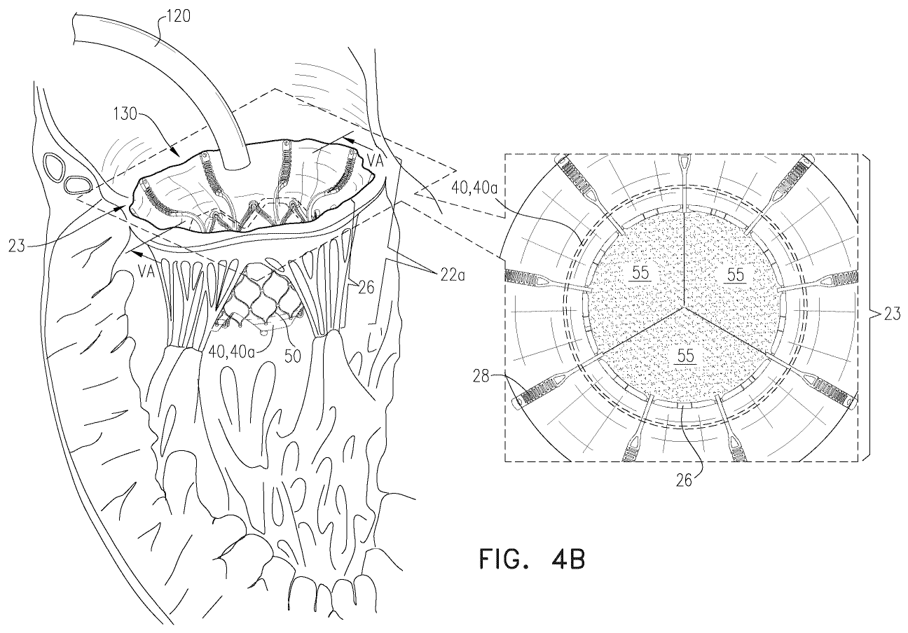

Reference is now made to FIGS. 4A-B, which are schematic illustrations of a system 110 showing a method of implanting prosthetic valve 22a at a calcified native valve 130, in accordance with some applications of the invention. Calcified native valve 130 is shown having calcified tissue 132, e.g., typically at native leaflets 131, which makes the valve annulus become less flexible and thicker. As shown, calcified tissue 132 is asymmetrically disposed at valve 130. Typically, prosthetic valve 22a is delivered toward in a compressed state within a catheter 120 toward calcified native valve 130, as shown in FIG. 4A. In FIG. 4B, prosthetic valve 22a is implanted an in an expanded state within calcified native valve 130. Catheter 120 is then retracted and withdrawn from the patient. Catheter 120 may be any suitable size. Valve 22a may be crimpable to a diameter of 7-14 mm, e.g., 9.45 mm, and as such, catheter 120 may comprise a 29 French catheter.

As shown in the bird's eye view of FIG. 4B, valve 22a has three prosthetic leaflets 55 which are disposed within the lumen of valve body 26 and are arranged to facilitate one-way upstream-to-downstream fluid flow through the lumen following implantation of prosthetic valve 22a in the heart of the patient. Annular element 23 functions as an upstream support portion at the atrial surface of calcified native valve 130 and anchors prosthetic valve 22a to prevent migration of valve 22a. Arms 28 are expanded and rest against the atrial surface of calcified native valve 130 anchoring prosthetic valve 22a to calcified native valve 130, thereby, arms 28 function as atrial anchoring arms. As is shown in FIGS. 5A-B, shape-conforming element 40 (1) expands in part within the calcified native valve 130 to fill gaps between the native valve and the prosthetic valve due to calcification, and (2) gets deformed in part by calcified tissue. Thus, shape-conforming element 40 conforms to the shape of the calcified tissue at calcified native valve 130.

Reference is now made to FIGS. 5A-B, which are schematic illustrations of prosthetic valve 22a implanted at calcified native valve 130, in accordance with some applications of the invention. As shown in the cross-sectional images, shape-conforming element 40 conforms to calcified tissue 132 of calcified native valve 130. Additionally, due to the spring quality and flexibility of outer flexible structure 50, structure 50 also conforms to calcified tissue 132 of calcified native valve 130, as shown, and enhances friction between prosthetic valve 22a and calcified native valve 130. For some applications of the present invention, outer flexible structure 50 partially conforms to calcified tissue 132 of calcified native valve 130 and enhances friction between prosthetic valve 22a and calcified native valve 130. For some applications of the present invention, outer flexible structure 50 does not conform to calcified tissue 132 of calcified native valve 130 and enhances friction between prosthetic valve 22a and calcified native valve 130. In such applications, the calcified tissue 132 may penetrate through lateral openings on outer flexible structure 50 such that shape-conforming element 40 conforms to the calcification while outer flexible structure 50 does not conform.

FIG. 5A shows prosthetic valve 22a with a part of shape-conforming element 40 and a part of frame 24 and a part of outer flexible structure 50 cut away for clarity of illustration.

It is with shape-conforming element 40 that the prosthetic valves described herein can stably fit within the native calcified valve 130 since shape-conforming element 40 has a sponge-like, spring-like, quality such that element 40 yields to or gives to physical force applied by calcified tissue against valves 22, 62, and 66. In such a manner, shape-conforming element 40 is able to conform to the topography of the calcified valve, which is typically an asymmetrical and abnormal topography. This conforming ability of shape-conforming element 40 enables gaps that would otherwise be created between frame 24 and calcified tissue 132 to be filled by shape-conforming element 40. As such, shape-conforming element 40 facilitates anchoring of valves 22, 62, and 66 to calcified tissue and/or to apparatus already implanted at the native valve.

Reference is now made to FIGS. 4B and 5A-B. It is to be noted that prosthetic valve 22a is shown partially covered in a fabric, for clarity of illustration. The scope of the present invention includes prosthetic valve 22a entirely covered by a fabric covering (not shown) which may comprise a single, unitary sheet of fabric or a plurality of sheets stitched together. The fabric covering may cover frame 24 and arms 28 in a manner in which shape-conforming element 40 is disposed on the outer surface of the fabric covering.

Reference is now made to FIG. 6A-B, which is a schematic illustration of a systems 150 and 151 comprising respective prosthetic valves 22d and 22′d in which shape-conforming element 40 comprises a longitudinally-shortened shape-conforming element 40d and 40′d, in accordance with some applications of the invention. It is to be noted that systems 150 and 151 are similar to and used in a similar fashion as systems 20, 60, 80, 90, and 100 described hereinabove, with the exception that prosthetic valves 22d and 22′d comprise longitudinally-shortened shape-conforming elements 40d and 40′d respectively, and like reference numbers refer to like parts. Typically, longitudinally-shortened shape-conforming element 40d of FIG. 6A has a height H2 of 3-7 mm, e.g., 5 mm, measured along longitudinal axis ax1 between an upstream surface at an upstream end 46 of shape-conforming element 40d and a downstream surface at a downstream end 48 of shape-conforming element 40d. Typically, longitudinally-shortened shape-conforming element 40′d of FIG. 6B has a height H3 of 5-12 mm, e.g., 7 mm, measured along longitudinal axis ax1 between an upstream surface at an upstream end 46 of shape-conforming element 40′d and a downstream surface at a downstream end 48 of shape-conforming element 40′d.

For such applications as shown in FIGS. 6A-B, longitudinally-shortened shape-conforming elements 40d and 40′d are not coupled to frame 24 axially along a longitudinal surface of frame 24, as other shape-conforming elements 40a, 40b, and 40c described herein. Rather, longitudinally-shortened shape-conforming elements 40d and 40′d are each coupled to frame 24 at an upstream section of frame 24, as shown. Alternatively, longitudinally-shortened shape-conforming elements 40d and 40′d may be coupled to frame 24 at a downstream section of frame 24 (not shown).

Longitudinally-shortened shape-conforming elements 40d and 40′d may be generally-rectangular, or ovoid, in cross-section. For some applications, longitudinally-shortened shape-conforming elements 40d and 40′d may be toroidal, annular, or ring shaped, and have a circular or oval cross-section. Longitudinally-shortened shape-conforming elements 40d and 40′d may be filled by a sponge, fabric, or foam, as described herein, or may be hollow, as is described hereinbelow with reference to FIG. 7.

As shown in FIG. 6A, longitudinally-shortened shape-conforming element 40d is shaped as a ring or a toroid and is positioned along a narrow portion of frame 24 that is downstream of arms 28 and at an upstream plane of valve body that is narrower than an intermediate downstream portion of valve body 26. That is, valve frame 24 forms a narrow waist 154 (or neck) at a portion of frame 24 at which longitudinally-shortened shape-conforming element 40d is coupled. This narrow waist 154 of frame 24 accommodates more shape-conforming element 40 than at wider intermediate portions downstream portions of frame 24.

As shown in FIGS. 6A-B, outer flexible structure 50 surrounds longitudinally-shortened shape-conforming elements 40d and 40′d. For some applications, as described hereinabove with reference system 60 of FIG. 1B, longitudinally-shortened shape-conforming elements 40d and 40′d are not surrounded by flexible structure 50. For applications in which longitudinally-shortened shape-conforming elements 40d and 40′d are surrounded by flexible structure 50, as shown, the downstream ends of flexible structure 50 may be fixedly coupled to the downstream end of frame 24. The downstream ends of flexible structure 50 may be fixedly coupled to the downstream end of frame 24 (1) by struts 51 (as shown) creating a space between the downstream end of structure 50 and the downstream end of frame 24, (2) by being directly coupled to frame 24 such as by welding, thereby eliminating a space between the downstream end of structure 50 and the downstream end of frame 24, or (3) by being coupled to frame 24 using sutures or other coupling elements. For some applications, the upstream end of structure 50 is not coupled to frame 24 (e.g., the upstream end of structure 50 rests against shape-conforming element 40, is sutured to shape-conforming element 40, or is loosely coupled to frame 24 by fabric (as shown in FIG. 10) Such coupling of the upstream end of structure 50 to either shape-conforming element 40 or to frame 24 by fabric enables axial sliding between outer flexible structure 50 and frame 24 and/or to shape-conforming element 40 disposed between structure 50 and frame 24, during transitioning of the prosthetic valve between the expanded and crimped states as frame 24 transitions between respective longitudinally shorter and longitudinally longer heights. It is to be noted that such configuration of the coupling of structure 50 to frame 24 described with reference to FIGS. 6A-B may be used in combination with any other prosthetic valve described herein.

FIG. 6B shows flexible structure 50 comprising a plurality of anchors 152 (e.g., barbs, teeth, or hooks) that extend and protrude out of a ventricular-facing surface of structure 50. Anchors 152 press into tissue of the calcified native valve thereby inhibiting movement of prosthetic valve 22 (in addition to inhibition of downstream movement provided by the geometry of the upstream support portion of annular element 23). For some applications, anchors 152 may be concentrated on structure 50 or may be sparsely positioned on structure 50. For some applications, anchors 152 may protrude radially and disposed circumferentially with respect to valve body 26 such that tissue of the native valve is captured between the anchors and the prosthetic valve by rotating the prosthetic valve about axis ax1. Anchors 152 are configured to enhance anchoring of valve 22′d to surrounding tissue. It is to be noted that anchors 152 may be coupled to any outer flexible structure 50 described herein.

Reference is now made to FIG. 7, which is a schematic illustration of a system 160 comprising a prosthetic valve 22e in which shape-conforming element 40 comprises a hollow shape-conforming element 40e, in accordance with some applications of the invention. It is to be noted that system 160 is similar to and used in a similar fashion as systems 20, 60, 64, 80, 90, and 100 described hereinabove, with the exception that prosthetic valve 22e comprises hollow shape-conforming element 40e, and like reference numbers refer to like parts.

Shape-conforming element 40 comprises an outer wall 161 that defines a radially inward lateral sections 43 and a radially outward lateral sections 41 which surround a hollow space 162. Typically, radially outward lateral section 41 is mechanically isolated from radially inward lateral section 43 such that during deformation of shape-conforming element 40, radially outward laterally section 41 changes shape while radially inward lateral section 43 remains undeformed.

Wall 161 typically comprises a braided mesh of biocompatible material, e.g., typically a metal such as nitinol. For some applications, wall 161 may comprise a thin fabric or foam or sponge that surrounds space 162.

FIG. 7 shows prosthetic valve 22e with a part of shape-conforming element 40 and a part of frame 24 and a part of outer flexible structure 50 cut away for clarity of illustration.

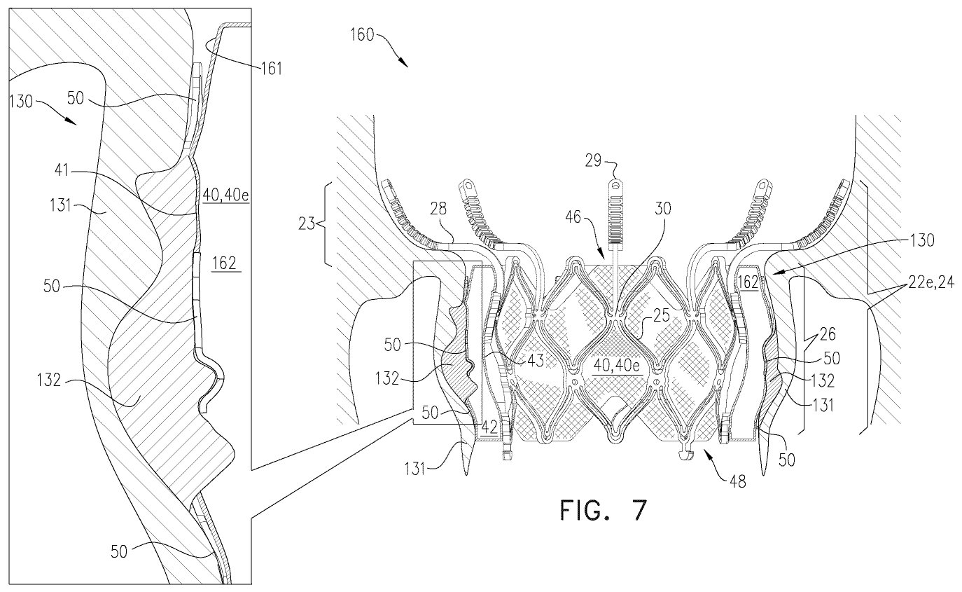

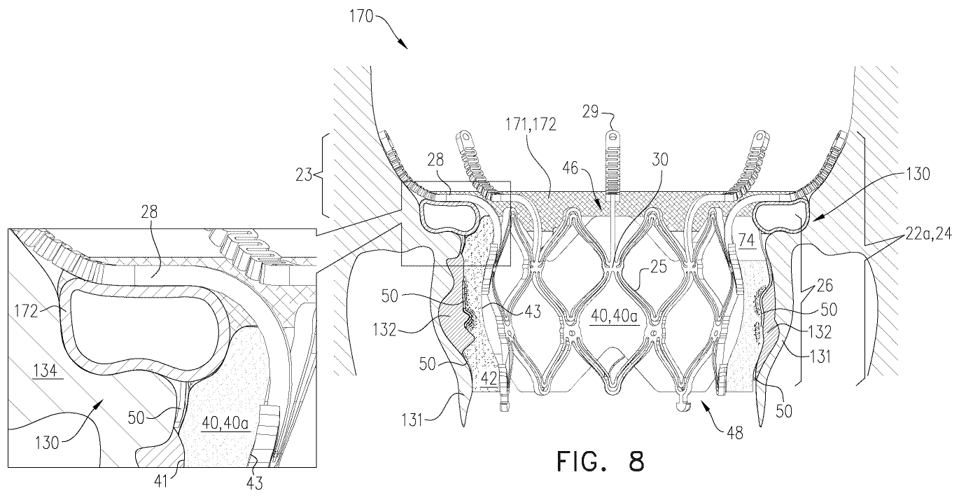

Reference is now made to FIG. 8, which is a schematic illustration of a system 170 comprising prosthetic valve 22a implanted at apparatus 171 pre-implanted at native valve 130, in accordance with some applications of the invention. As shown, the pre-implanted apparatus 171 comprises an annuloplasty structure 172, e.g., a full annuloplasty ring or partial annuloplasty ring, that is implanted at an annulus 134 of native valve 130. It is to be noted that shape-conforming element 40 of prosthetic valve 22a conforms to the shape of annuloplasty structure 172 already pre-implanted at valve 130 and/or to fibrotic tissue that grows around structure 172. It is to be noted that prosthetic valve 22a may conform to any apparatus 171 pre-implanted at native valve 130. It is to be noted that system 170 is similar to and used in a similar fashion as systems 20, 60, 64, 80, 90, 100, 150, 151, and 160 described hereinabove, and like reference numbers refer to like parts.

FIG. 8 shows annuloplasty structure 172 and prosthetic valve 22a with a part of shape-conforming element 40 and a part of frame 24 and a part of outer flexible structure 50 cut away for clarity of illustration.

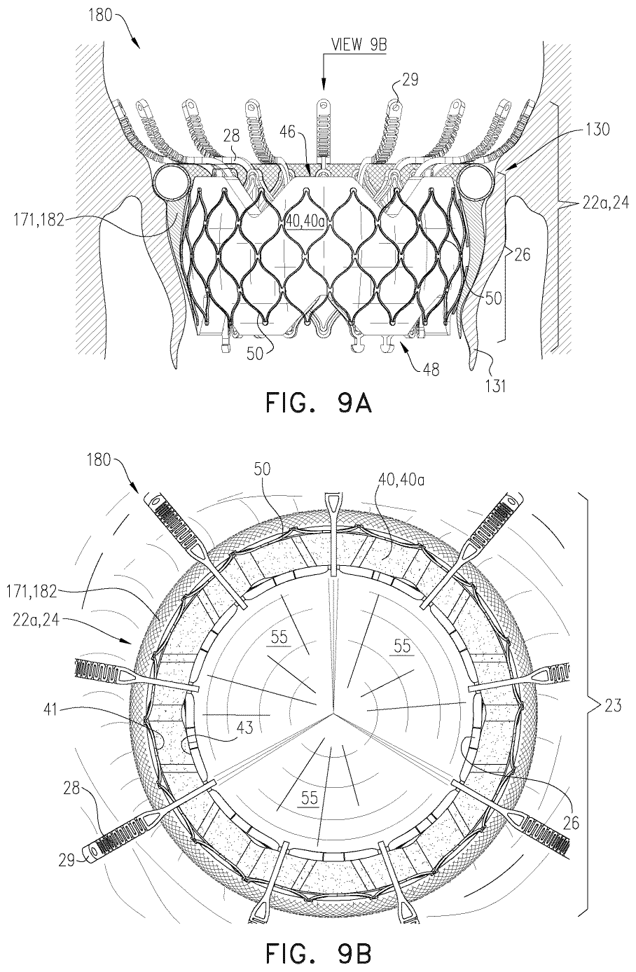

Reference is now made to FIGS. 9A-B, which are schematic illustrations of a system 180 comprising prosthetic valve 22a implanted at apparatus 171 pre-implanted at native valve 130, in accordance with some applications of the invention. As shown, the pre-implanted apparatus 171 comprises a prosthetic valve 182. Pre-implanted prosthetic valve 182 may comprise a transcatheter valve or a surgical valve. It is to be noted that shape-conforming element 40 of prosthetic valve 22a conforms to the shape of prosthetic valve 182 already pre-implanted at valve 130 and/or to fibrotic tissue that grows around valve 182. In such cases, pre-implanted prosthetic valve 182 may comprise a prosthetic valve that is not functioning properly. It is to be noted that prosthetic valve 22a may conform to any apparatus pre-implanted at native valve 130. It is to be noted that system 180 is similar to and used in a similar fashion as systems 20, 60, 64, 80, 90, 100, 150, 151, and 160 described hereinabove, and like reference numbers refer to like parts.

As shown in the bird's eye view of FIG. 9B, valve 22a has three prosthetic leaflets 55 which are disposed within the lumen of valve body 26 and are arranged to facilitate one-way upstream-to-downstream fluid flow through the lumen following implantation of prosthetic valve 22a in the heart of the patient.

Reference is now made to FIGS. 8 and 9A-B. Prosthetic valve 22a may be coupled to the pre-implanted apparatus in the presence or absence of calcified tissue 132 at native valve 130. It is to be noted that although prosthetic valve 22a is shown implanted at apparatus 171 pre-implanted at native valve 130, any one of prosthetic valves 22b, 22c, 22d, 22′d, 22e, 62, 66 described herein may be implanted at apparatus 171 pre-implanted at native valve 130.

Reference is now made to FIG. 10, which is a schematic illustration of a system 190 for use at a calcified native heart valve of a patient and comprising prosthetic valve 66 described hereinabove with reference to FIG. 1C, in accordance with some applications of the invention. Valve 66 comprises a prosthetic valve 66′ comprising a flexible sheet 192, e.g., a fabric. For some applications flexible sheet 192 comprises a single, unitary sheet of fabric or a plurality of sheets stitched together. For some arms 28 are covered with a covering, e.g., extending between arms 28 to form an annular shape. It is hypothesized that this reduces a likelihood of paravalvular leakage. For such embodiments, excess covering may be provided between arms 28 of the upstream support portion, so as to facilitate their independent movement. The covering can cover the atrial, upstream side of the upstream support portion (i.e., the plurality of arms 28), and may additionally or alternatively cover the ventricular, downstream side of the upstream support portion. For example, the covering may extend over tips 29 of arms 28 and down the outside of the arms, or a separate piece of covering may be provided on the ventricular, downstream side of the upstream support portion.

As described hereinabove with reference to FIG. 1C, upstream ends 53 of outer flexible structure 50b is coupled to frame 24 by flexible sheet 192, e.g., fabric. As shown, flexible sheet 192 comprises excess portions 194 of material of sheet 192 which enable valve 66′ to transition between the expanded and crimped states as frame 24 transitions between respective longitudinally shorter and longitudinally longer heights. When valve 66′ is crimped, sheet 192 and thereby portions 194 are pulled tighter, and when valve 66′ is expanded, as shown, portions 194 of sheet 192 are slackened.

Sheet 192 comprises a flexible material, such as a fabric, e.g., including polyester. For some applications, sheet 192 comprises a braided mesh. Typically, but not necessarily, sheet 192 comprises polyethylene terephthalate (commonly known as DACRON™).

For some applications, as shown in FIG. 10, bulging outer flexible structure 50b does not comprise upstream narrow portion 67 and/or upstream ends 53 are not fixedly coupled to upstream end 32 of valve body 26 of frame 24. In such applications, upstream ends 53 slide longitudinally with respect to upstream end 32 of valve body 26 of frame 24 during expanding and contracting of the prosthetic valve.

Intermediate wide portion 68 typically does not contact an outer surface of valve body 26. When conforming to the shape of the calcified valve and/or to apparatus already implanted at the native valve, intermediate wide portion 68 can move toward the outer surface of valve body 26 by being 20-90%, e.g., by 60-70%, radially compressed from a resting state portion 68. Typically, intermediate wide portion 68 undergoes a radial deformation of between 1 and 9 mm, e.g., 5 mm, during implantation.

For some applications, outer flexible structure 50b includes a plurality of anchors (e.g., barbs, teeth, or hooks) (not shown but, for example, anchors 152 shown hereinbelow with reference to FIG. 6B) that extend and protrude out of a ventricular-facing surface of structure 50b. The anchors press into tissue of the calcified native valve thereby inhibiting movement of prosthetic valve 66 (in addition to inhibition of downstream movement provided by the geometry of the upstream support portion of annular element 23). For some applications, the anchors may be concentrated on structure 50b or may be sparsely positioned on structure 50b. For some applications, the anchors may protrude radially and disposed circumferentially with respect to valve body 26 such that tissue of the native valve is captured between the anchors and the prosthetic valve by rotating the prosthetic valve about axis ax1.

Reference is now made to FIGS. 6A and 10. It is to be noted that for some applications of the present invention, prosthetic valve 66 of FIG. 10 may comprise longitudinally-shortened shape-conforming element 40d described hereinabove with reference to FIG. 6A at narrow waist 154 of valve 66.

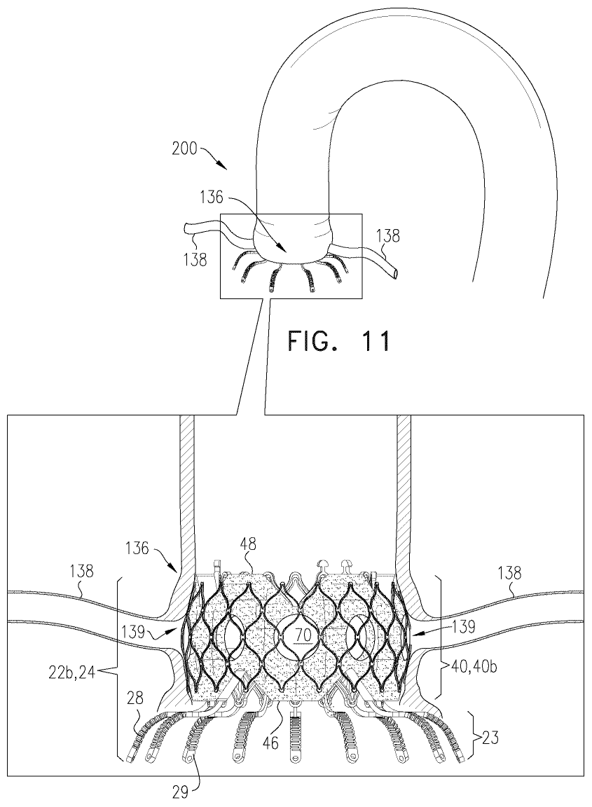

Reference is now made to FIG. 11, which is a schematic illustration of a system 200 for use at a calcified native aortic heart valve 136 of a patient, in accordance with some applications of the invention. The scope of the present invention includes a prosthetic valve for direct implantation in native aortic valve 136 or in apparatus pre-implanted at native aortic valve 136.

For some applications, prosthetic valve 22b as described hereinabove with reference to FIG. 3B is implanted in aortic heart valve 136, as shown. It is to be noted that any prosthetic valve described herein may be implanted in aortic heart valve 136, e.g., prosthetic valve 22c as described hereinabove with reference to FIG. 3C, or prosthetic valves 66 or 66′ as described hereinabove with reference to FIGS. 1C and 10. For some applications of the present invention, for prosthetic valves shown herein for implantation at native aortic valve 136 or in apparatus pre-implanted at native aortic valve 136, the prosthetic valve may be shorter than the prosthetic valves described herein so as to not to interfere with the left ventricular outflow tract nor with the coronary arteries 138 and the coronary ostia 139. In such applications for which prosthetic valve 22b is implanted at native aortic valve 136, windows 70 through wall 45 of shape-conforming element 40 minimize the overall profile of valve 22b and minimizes or eliminates obstruction of coronary ostia 139 because following implantation of valve 22b or 22c, windows 70 are aligned with coronary ostia 139 to facilitate unobstructed blood flow through coronary ostia 139.

FIG. 11 shows valve 22b without covering 44 described hereinabove, for clarity of illustration.

Reference is now made to FIGS. 1A-C, 2A-C, 3A-C, 4A-B, 5A-B, and 6A-B, 7-11. It is to be noted that although prosthetic valve 22a is shown implanted at native valve 130 in FIGS. 4A-B, 5A-B, and 8-9, any one of prosthetic valves 22b, 22c, 22d, 22′d, 22e, 62, 66 described herein may be implanted at calcified native valve 130 and/or at apparatus already implanted at native valve 130. It is to be noted that shape-conforming elements 40 and/or outer flexible structures 50 described herein may be implanted at any suitable location in the body, in particular, in stenosed and/or calcified areas. Additionally, it is to be noted that prosthetic valves 22a, 22b, 22c, 22d, 22′d, 22e, 62, or 66 described herein are shown without an outer fabric covering, for clarity of illustration. It is to be noted that the scope of the present invention includes prosthetic valves 22a, 22b, 22c, 22d, 22′d, 22e, 62, or 66 that are partially or fully covered by an outer covering.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use at a calcified native valve of a heart of a patient, the apparatus comprising a prosthetic valve deliverable to the heart through a catheter, the prosthetic valve comprising:
(a) a frame that comprises:
a valve body that circumscribes a central longitudinal axis of the prosthetic valve and defines a lumen along the axis; and
a plurality of arms that are attached to and extend from respective circumferential sites of the valve body in an upstream direction, each of the arms configured to extend radially outward to a respective arm-tip in an expanded state of the frame;
(b) a plurality of prosthetic leaflets, disposed within the lumen, and arranged to facilitate one-way upstream-to-downstream fluid flow through the lumen following implantation of the prosthetic valve in the heart; and
(c) a shape-conforming element surrounding the valve body, the shape-conforming element configured to conform to calcified areas of cardiac tissue of the calcified native heart valve.

2. Apparatus comprising a prosthetic valve deliverable through a catheter to a native valve of a heart of a patient, the prosthetic valve comprising:
(a) a frame that comprises:
a valve body that circumscribes a central longitudinal axis of the prosthetic valve and defines a lumen along the axis; and
a plurality of arms that are attached to and extend from respective circumferential sites of the valve body in an upstream direction, each of the arms configured to extend radially outward to a respective arm-tip in an expanded state of the frame;
(b) a plurality of prosthetic leaflets, disposed within the lumen, and arranged to facilitate one-way upstream-to-downstream fluid flow through the lumen following implantation of the prosthetic valve in the heart; and
(c) a shape-conforming element surrounding the valve body, the shape-conforming element being configured to conform to apparatus previously implanted at the native heart valve.

3. (canceled)

4. The apparatus according to claim 1, wherein the shape-conforming element comprises a metal wall surrounding a hollow space.

5-6. (canceled)

7. The apparatus according to claim 1, wherein the plurality of arms comprises 3-24 arms.

8. The apparatus according to claim 1, wherein each arm, when extending radially outward from the valve body, has a radially-extending portion having a downstream surface having a downstream-directed convex portion adjacent the arm-tip.

9. The apparatus according to claim 8, wherein, in the expanded state of the frame, (1) each arm has a downstream-directed concave portion at a radially-inner portion of the arm, and (2) the downstream-directed convex portion of each arm is between the downstream-directed concave portion and the arm-tip.

10. The apparatus according to claim 9, wherein the downstream-directed convex portion is upstream of an upstream end of the shape-conforming element.

11-13. (canceled)

14. The apparatus according to claim 1, wherein the shape-conforming element comprises a sponge.

15-19. (canceled)

20. The apparatus according to claim 1, wherein the shape-conforming element comprises a shape memory polymer.

21. The apparatus according to claim 1, wherein the shape-conforming element undergoes a radial deformation of between 1 and 9 mm.

22-25. (canceled)

26. The apparatus according to claim 1, wherein the shape-conforming element has a strain at break of 20-80%.

27-28. (canceled)

29. The apparatus according to claim 1, wherein the shape-conforming element has a height of 5-20 mm measured along the central longitudinal axis of the prosthetic valve.

30. The apparatus according to claim 1, wherein the shape-conforming element covers between 15-95% of the valve body.

31-35. (canceled)

36. The apparatus according to claim 1, wherein the prosthetic valve further comprises an outer flexible structure surrounding the shape-conforming element and configured to enhance friction between the prosthetic valve and the calcified native heart valve.

37-42. (canceled)

43. The apparatus according to claim 1, wherein at least one surface of the shape-conforming element is arranged in a zig-zag pattern around an outer surface of the valve body.

44. The apparatus according to claim 43, wherein the at least one surface of the shape-conforming element comprises:
an upstream surface of the shape-conforming element that is arranged in a zig-zag pattern; and
a downstream surface of the shape-conforming element that is arranged in a zig-zag pattern.

45. The apparatus according to claim 44, wherein the zig-zag pattern of the upstream surface is rotationally offset with respect to the zig-zag pattern of the downstream surface.

46. The apparatus according to claim 43, wherein the shape-conforming element is shaped so as to define:
an upstream end having a plurality of upstream surfaces alternating with a plurality of downstream-directed cutout portions, each downstream-directed cutout portion having respective descending and ascending edges connected at a downstream-directed vertex, and
a downstream end having a plurality of downstream surfaces alternating with a plurality of upstream-directed cutout portions, each upstream-directed cutout portion having respective ascending and descending edges connected at an upstream-directed vertex.

47. (canceled)

48. The apparatus according to claim 46, wherein the downstream surfaces are offset with respect to the upstream surfaces and the downstream-directed vertices are offset with respect to the upstream-directed vertices.

49. The apparatus according to claim 48, wherein:
each downstream-directed vertex faces a midpoint of an opposite downstream surface of the downstream end of the shape-conforming element, and
each upstream-directed vertex faces a midpoint of an opposite upstream surface of the upstream end of the shape-conforming element.

50-70. (canceled)
